(19) United States
(12) Patent Application Publication
(10) Pub. No.: US 2023/0364173 A1
(43) Pub. Date: Nov. 16, 2023

(54) SYNERGISTIC HERBAL COMPOSITIONS FOR IMMUNITY, COLD AND COUGH

(71) Applicant: LAILA NUTRACEUTICALS, Vijayawada (IN)

(72) Inventors: Ganga Raju Gokaraju, Vijayawada (IN); Venkata Kanaka Ranga Raju Gokaraju, Vijayawada (IN); Kiran Bhupathiraju, Vijayawada (IN); Rama Raju Gokaraju, Vijayawada (IN); Venkateswarlu Somepalli, Vijayawada (IN); Trimurtulu Golakoti, Vijayawada (IN); Krishanu Senguptha, Vijayawada (IN); Venkata Krishna Raju Alluri, Vijayawada (IN)

(21) Appl. No.: 18/246,768

(22) PCT Filed: Sep. 24, 2021
(86) PCT No.: PCT/IN2021/050937

§ 371 Date: Mar. 27, 2023

(30) Foreign Application Priority Data
Sep. 25, 2020 (IN) 202041041718

(51) Int. Cl. A61K 36/484; A61K 36/59; A61K 36/9068; A61K 36/19; A61K 36/53; A61K 33/30; A61P 37/02

(52) U.S. Cl. CPC A61K 36/484; A61K 36/59; A61K 36/9068; A61K 36/19; A61K 36/53; A61K 33/30; A61P 37/02; A61K 2236/00

(57) ABSTRACT

The present invention discloses synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; process for their preparation, methods of treatment and use of such compositions for improving immunity and improving respiratory health that include allergic airway inflammation, cold and cough.

### TECHNICAL FIELD OF THE INVENTION

The invention relates to synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof, and a second ingredient selected from the extract, fraction, phytochemical or mixtures thereof derived from Tinospora cordifolia, Zingiber officinale, Justicia adhatoda, and Ocimum sanctum; for obtaining at least one health benefit selected from improving immunity and improving respiratory health that include allergic airway inflammation, cold and cough.

### BACKGROUND OF THE INVENTION

Immunity: Immunity is the body's natural defense system against various bacterial/viral/fungal infections, diseases, and different environmental stimuli. Primarily, host immunity is classified into innate and adaptive immune responses. Innate immune responses are rapid and non-specific to pathogens, which are mediated by innate immune cells such as myeloid cells, natural killer (NK) cells, innate lymphoid cells, and humoral systems such as defensins and complement systems. Adaptive immune responses are slower but specific to the infections with the recruitment of B- and T-lymphocytes and generation of long-lived immunological memory. Recent research has demonstrated the strong coordinated network between the innate and adaptive immune systems to effectively tackle the infections through bidirectional activation through eliciting innate immune responses such as phagocytosis and B/T-cell-mediated adaptive immune responses. However, if these immune responses are excessively or inappropriately activated, autoimmune disorders like rheumatoid arthritis, allergies, and cancers may develop.

Respiratory health: The lungs are constantly exposed to the external environment and come in contact with potential air-borne pathogens, allergens, and environmental pollutants. This constant exposure requires an effective and fast-acting immune system. Hence, the pulmonary immune system contains a broad armamentarium of cellular and humoral defense mechanisms in the airways. The coordinated and complex interplay between the resident airway epithelial cells and infiltrating immune cells along with the secretions of defensins, mucins, or collectins shape the outcome of host-pathogen, host-allergen, host-particle interactions within the airway microenvironment. These interactions further activate the downstream immune responses through the release of mediators such as chemokines (CCL-2, CCL-20), cytokines (IL-1α, IL-1β), and lipid mediators (eicosanoids/leukotrienes). Conversely, excessive immunological tolerance in the airways may lead to an ineffective clearance of infectious agents such as influenza, tuberculosis, respiratory syncytial virus, Streptococcus pneumoniae, or coronavirus, which may lead to inflammatory lung disease, bronchitis, pneumonia, and sepsis.

Allergic airway inflammation: Airway inflammation, including asthma and allergic rhinitis, is the common chronic respiratory illness worldwide. Low temperature, accompanying low air humidity, air pollutants (such as pollen, dust, etc.) affect the respiratory epithelium and induce hyper responsiveness (bronchial allergy) and narrowing of the respiratory airways. Poor air quality and cold air also influence chronic airway inflammation, which is likely to aggravate respiratory symptoms. Epidemiological studies have shown that respiratory symptoms such as wheezing, cough, runny, stuffy nose, congestion, phlegm (a byproduct of inflammation in the sinuses and the lungs) are common among adults and children. A decline in lung function and a high prevalence of respiratory symptoms in the cold indoor environment have also been reported. Allergic airway inflammation is characterized by reversible airway obstruction, airway hyper-responsiveness (AHR), infiltration of eosinophils and type 2 T-cells (that activate eosinophils and mast cells) into the airway submucosa, mucus hypersecretion, and airway remodeling. In the hypersensitivity (allergic) reaction, the allergen-specific immunoglobulins of the IgE class bound to the surfaces of basophils and mast cells present in the sub-epithelial layer of the airways. These bound IgE molecules immediately release leukotrienes, prostaglandins, and histamine. These mediators contract airway smooth muscle cells and induce edema and mucus secretion, leading to narrowed, constricted airways. Locally produced chemokines stimulate the recruitment of eosinophils, macrophages, neutrophils, and T-lymphocytes in the airways.

Common cold: Common cold (synonyms: acute coryza, acute viral nasopharyngitis) is a highly contagious viral disease of the upper respiratory tract involving the nose, sinuses, pharynx, and larynx. At least 200 identified viruses are capable of causing the common cold. Among these, rhinoviruses, coronaviruses, parainfluenza viruses, respiratory syncytial virus, adenoviruses, and enteroviruses are common. The histologic effects of infection vary from mild to severe epithelial destruction. The infection causes vasodilation and hypersecretion. The common clinical symptoms include nasal congestion, nasal discharge, postnasal drip (PND), throat clearing, sneezing, and cough.

Cough: Cough is a forced expulsive maneuver, usually against a closed glottis and which is associated with a characteristic sound. An acute cough is defined as one lasting less than 3 weeks. It is the commonest symptom associated with acute exacerbations and hospitalizations with asthma and chronic obstructive pulmonary disease (COPD). Chronic cough is defined as one lasting more than 8 weeks. Most patients present with a dry or minimally productive cough. Because of the variable and episodic nature of acute cough, cough suppressants such as dextromethorphan, menthol, sedative antihistamines, codeine or pholcodine are used.

Current synthetic drugs hold no promise in the complete healing of these disorders and also they have some safety concerns. Thus there is a scope for the provision of safe and potent herbal formulations having built-in immune-stimulating and inflammation-modulating effects to viral respiratory infections while still helping the immune system cope better with the infections.

Patent publication U.S. Pat. No. 6,979,471B1 disclosed a composition comprising a bio-enhancer or bioavailability facilitator selected from the group consisting of isolated and purified glycyrrhizic acid, isolated and purified glycyrrhizin or mixtures thereof in a concentration of about 1 μg/ml and an agent selected from one or more nutraceuticals, antibiotics, anti-infective agents, and anti-cancer agents.

Another patent publication US20030228383A1 disclosed a herbal composition for the treatment of chronic respiratory disorders such as cold, cough, allergic asthma, seasonal allergic rhinitis, pharyngitis, laryngitis comprises extracts derived from Ayurvedic plants selected from the Ocimum sanctum, Glycyrrhiza glabra, Curcuma longa, Zingiber officinale, Adhatoda vasika, Solanum indicum, Saussurea lappa, Piper cubeba, Terminalia bellirica, Aloe barbadensis, Inula racemosa, Clerodendrum serratum, Solanum xanthocorpum, Pipper longum, Alpinia galangal, Terminalia chebula, Emblica officinalis and a process for preparing the same.

Another patent publication EP1529445B1 disclosed a dietary supplement composition comprising an active ingredient, a vegetable protein containing carrier, a vegetable source for sugar, a vegetable source for fat, and salt, wherein the active ingredient is constituted by a mixture of 14-26% of Zingiber officinalis, 14-26% of Piper longum, 14-26% of Piper nigrum, 14-26% of Cucumis trigonus, and 14-26% of Glycyrrhiza glabra.

Patent publications CN1010373B, CN104530177B, CN105541956A and CN1120172C disclosed various process techniques for the preparation of licorzinc (zinc glycyrrhizinate) from glycyrrhizic acid.

To the best of inventor's knowledge, synergistic herbal compositions comprising a first ingredient zinc/potassium double salt of glycyrrhizin derived from Glycyrrhiza glabra; and a second ingredient selected from the extract, fraction, phytochemical or mixtures thereof derived from Tinospora cordifolia, Zingiber officinale, Justicia adhatoda, and Ocimum sanctum are not known in the literature.

So there is a continuous need in the art to provide potent natural Glycyrrhiza glabra extract salts and their compositions for boosting immune function, rejuvenating the immune system, regulating the immune system, improving lung function, supporting respiratory health, and treating/alleviating symptoms associated with cold, cough, flu, runny nose and sinus symptoms.

### Objective of the Invention

Therefore, the main object of the present invention is to provide synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

Another objective of the invention is to provide method of obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose in a human; wherein the method comprises supplementing the said human with an effective dose of a composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof, and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

Yet another objective of the invention is to provide the use of a synergistic herbal composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

### SUMMARY OF THE INVENTION

The present invention provides synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof, and a second ingredient selected from extract, fraction, phytochemical or mixtures thereof derived from Tinospora cordifolia, Zingiber officinale, Justicia adhatoda, and Ocimum sanctum; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

Another aspect of the invention provides synergistic herbal compositions comprising a first ingredient, Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

In a further aspect, the invention provides a process for the preparation of the compositions comprising a first ingredient, Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

In yet another aspect, the invention provides method of obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose in a human; wherein the method comprises supplementing the said human with an effective dose of a composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

In a further aspect, the invention provides the use of a synergistic herbal composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

### DETAILED DESCRIPTION OF THE INVENTION

The invention will now be described in detail in connection with certain preferred and optional embodiments so that various aspects thereof may be more fully understood and appreciated.

The terms glycyrrhizin and glycyrrhizic acid are the same and are interchangeable. Thus the statements “zinc/potassium double salt of glycyrrhizin, zinc/potassium double salt of glycyrrhizic acid and zinc/potassium double salt of glycyrrhizinate” made in the specification convey the same meaning and are interchangeable. Similarly, zinc salt of glycyrrhizin, zinc salt of glycyrrhizic acid and zinc salt of glycyrrhizinate; potassium salt of glycyrrhizin, potassium salt of glycyrrhizic acid and potassium salt of glycyrrhizinate; also conveys the same meaning and are used interchangeably in the specification.

The terms “improve”, “ameliorate,” and “better” as used herein, conveys the same meaning and are interchangeable. Unless stated to the contrary, any of the words, “including”, “includes”, “comprising”, and comprises” mean “including without limitation” and shall not be construed to limit any general statement that it follows to the specific or similar items. The terms “metal salt”, “metal complex” and “metal chelate” also convey the same meaning and are used interchangeably in the specification. Unless specified otherwise, all technical and scientific terms used herein have the same meaning as commonly understood by one of ordinary skill in the art to which this invention belongs.

The source of the herbs used in the invention is as follows:
1. Glycyrrhiza glabra was procured from the local market.
2. Justicia adhatoda was collected from Hanumanthulagudem village, Hanumanthulagudem Panchayati, Nuzividu Mandal, Krishna District, Andhra Pradesh and it was wild.
3. Zingiber officinale was collected from Penubaka village, Penubaka Panchayati, Tadepalli Mandal, Guntur District, Andhra Pradesh, and it was cultivated.
4. Tinospora cordifolia was collected from Mulapadu village, Mulapadu Panchayati, Ibrahimpatnam Mandal, Krishna District, Andhra Pradesh, and it was wild.
5. Ocimum sanctum was collected from Kondapalli village, Ibrahimpatnam Panchayati, Ibrahimpatnam Mandal, Krishna District, Andhra Pradesh, and it was wild.

Glycyrrhiza glabra: G. glabra is a widely used and extensively researched medicinal plant around the world. In Ayurveda, it is used for treating coughs, colds, and chills. In traditional medicine, its roots have been used for treating chest and lung diseases, pneumonia, bronchitis, arthritis, bronchial asthma, kidney diseases, heart diseases, gastric ulcer, mouth ulcers, coughs, swellings, excessive salivation, fluid retention, low blood pressure, allergies, catarrhs of the upper respiratory tract, liver toxicity, hyperglycemia, and certain viral infections. Glycyrrhiza glabra contains several phytochemical constituents and out of which glycyrrhizin (glycyrrhizic acid) is the major active phytochemical.

Glycyrrhizin: Glycyrrhizin, also called as glycyrrhizic acid, is a triterpenoid saponin obtained from the root and rhizome extracts of Glycyrrhiza glabra (Licorice). Glycyrrhizic acid is the major characteristic constituent of licorice, which is primarily responsible for its pharmacological activities and sweet taste. It is widely used in traditional Indian, Chinese, and Tibetan medicinal preparations. Glycyrrhizic acid is also used as a sweetener in many applications, and it is reported to be 30-50 times sweeter than sucrose. Pharmacologically, glycyrrhizin is used as an anti-inflammatory, anti-thrombin, anti-tumor, anti-ulcer, anti-allergic, anti-oxidant, anti-viral agent, and also for treating chronic hepatic diseases. The chemical structure of glycyrrhizin is shown below (FIG. 1).

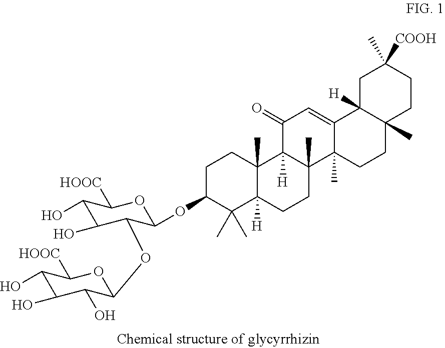

Zinc is the second most abundantly distributed trace element in the body after iron. It is found only in modest amounts in varieties of food, such as beef, poultry, seafood, and grains. It is needed for wound healing, sense of taste and smell, DNA synthesis, and it supports normal growth and development during pregnancy, childhood, and adolescence. Zinc deficiency in the body can lead to a variety of health-related problems. Zinc is essential for the biological activity of over 300 enzymes that aid in metabolism, digestion, nerve function, and many other processes. Zn is known to be important in immune function, DNA and protein production, and cell division.

Potassium is the essential mineral needed in higher quantity than any other metal, with a requirement of up to 3500 mg per day. Potassium plays a vital role in blood pressure regulation, carbohydrate metabolism, and fluid balance. The functions of potassium and sodium in living organisms are quite different. Potassium is the major cation present inside the animal cells, while sodium is the major cation present outside the animal cells, and they play a critical role in maintaining membrane potential. As sodium consumption rises, increased potassium is needed to balance the effect of sodium on blood pressure.

The inventors surprisingly found that the zinc/potassium double salt of glycyrrhizin derived from Glycyrrhiza glabra is useful to improve immunity, lung function, respiratory health, and treating/alleviating symptoms associated with cold and cough. Zinc/potassium double salt of glycyrrhizin derived from Glycyrrhiza glabra is not known in the literature.

Therefore, the present invention provides synergistic herbal compositions comprising a first ingredient zinc/potassium double salt of glycyrrhizin derived from Glycyrrhiza glabra; and a second ingredient selected from the extract, fraction, phytochemical or mixtures thereof derived from Tinospora cordifolia, Zingiber officinale, Justicia adhatoda and Ocimum sanctum; further containing optionally at least one component selected from pharmaceutically or nutraceutically or dietically acceptable excipients, carriers and diluents.

After several experimental trails, the inventors achieved the preparation of zinc/potassium double salt of glycyrrhizin derived from Glycyrrhiza glabra; by two methods, (i) neutralization of G. glabra extract with zinc oxide and potassium carbonate; and (ii) G. glabra extract was purified by cation exchange resin and the glycyrrhizin thus obtained was neutralized with zinc oxide and potassium carbonate.

Thus, the 50% aqueous ethanol extract of Glycyrrhiza glabra was neutralized with metal oxide such as zinc oxide followed by metal carbonate such as potassium carbonate or metal hydroxide such as potassium hydroxide or mixtures thereof to yield zinc/potassium double salt of glycyrrhizin (Gly-2). The extract was analyzed for glycyrrhizin by the HPLC method of analysis and found that the extract contains 14.88% of glycyrrhizin. Zinc and potassium were estimated by ICP mass and found that the extract contains 0.9% of zinc and 2.5% of potassium (Example 1).

In the second method, purified glycyrrhizic acid was obtained by purification of 50% aqueous ethanol extract of Glycyrrhiza glabra root through a cation exchange resin column. The glycyrrhizic acid thus obtained was neutralized with metal oxide followed by metal carbonate or metal hydroxide or mixtures thereof to yield zinc/potassium double salt of glycyrrhizin (Gly-3).

Similarly, zinc salt of glycyrrhizin derived from G. glabra (Gly-4); and potassium salt of glycyrrhizin derived from G. glabra (Gly-5) were also prepared. For comparison, 50% aqueous ethanol extract of G. glabra was also prepared (Gly-1). These extracts were analyzed for glycyrrhizin by HPLC. Zinc and potassium by ICP mass and the results are summarized in Table-3.

Salt formation: The chemical structure of glycyrrhizic acid contains three carboxylic acid groups, which are capable of forming salts with bases. Glycyrrhiza glabra extract containing glycyrrhizic acid when treated with metal oxides, metal hydroxides, and metal carbonates etc., forms metal salt of glycyrrhizic acid. For example, neutralization of Glycyrrhiza glabra extract with zinc oxide and potassium carbonate followed by filtration of the solution to remove water insoluble and unreacted zinc oxide gave Glycyrrhiza glabra extract containing zinc/potassium double salt of glycyrrhizic acid. The presence of zinc and potassium by ICP-MS analysis of the extract clearly indicates the formation of salt.

To address the problem and to provide safe herbal composition(s) for improving immunity, respiratory health that include allergic airway inflammation, cold, and cough, the following cell-based assays have been conducted to evaluate the efficacy of the compositions in boosting immune function, respiratory health that include allergic airway inflammation, cold, and cough.
(i) Interferon-γ (IFN-γ) production
(ii) Interleukin-2 (IL-2) production
(iii) Leukotriene B₄ (LTB₄) inhibition
(iv) Histamine assay

Interferon-γ: INF-γ is a cytokine produced mainly by Th1 lymphocytes in response to microbes, intracellular parasites, viruses, and autoimmune reactions. Interferon-γ (IFN-γ) has been shown to have profound effects on both innate and adaptive immunity, which contribute to host protection. IFN-γ is produced by adaptive CD4+ Th1 T cells, CD8+ cytotoxic T cells, natural killer (NK) cells, B cells, NKT cells, and innate professional antigen-presenting cells (APCs) [monocyte/macrophage, dendritic cells (DC)]. IFN-γ has a critical role in recognizing and eliminating pathogens as it can coordinate a plethora of anti-microbial and anti-viral functions through cell-mediated immunity. It can enhance the antigen recognizing capacity of APCs and amplify their antigen presentation to the T cells, subsequently increasing the production of reactive oxygen species (ROS) and reactive nitrogen intermediates (RNIs) and induce anti-viral responses. In the context of viral infections, INF-γ treatment protects neurons from varicella-zoster virus and limit Hepatitis C virus proliferation in HIV patients. In this regard, compounds promoting INF-γ production would be important to boost the host immune system and also for improving respiratory health.

Interleukin-2: Interleukin-2 (IL-2) was one of the first cytokines discovered. It can promote T cell proliferation and differentiation in vitro and plays a crucial role during antigen-driven clonal expansion of T cells in vivo. With respect to immune activation in vivo, IL-2 has a role in the proliferation and survival of T cells and differentiation of T cells into effector T cells. In the case of chronic infection, IL-2 is also an important factor in generating memory T cells that can undergo secondary expansion when they re-encounter the antigen. Alternatively, IL-2 can promote activation-induced cell death (AICD) of the T cells, down-regulating the immune response after the clonal expansion of antigen-specific T cells. IL-2 can also prime CD8+ T cells with non-infectious immunogens. Conversely, very high levels of IL-2 can bind to CD25 and differentiate the T cells into regulatory T cells (Tregs), which will suppress the excessive immune response. In this regard, compounds promoting IL-2 production would be important to boost the host immune system.

Leukotriene B₄ (LTB₄): Leukotriene B₄ is a metabolite of the 5-lipoxygenase pathway and was discovered in 1979. The leukotrienes (LTs), a family of proinflammatory lipid mediators, play an important role in the pathogenesis of allergic inflammation and are divided into two classes: the chemoattractant LTB₄ and the spasmogenic cysteinyl LTs [CysLTs: LTC₄, LTD₄, and LTE₄]. LTB₄ cannot be stored and released, but it is synthesized from arachidonic acid in activated innate immune cells such as granulocytes, macrophages, and mast cells following several enzymatic steps. Three distinct receptors for LTB₄ are PPARα, BLT1, and BLT2. LTB₄ is closely related to the pathogenesis of several allergic diseases, including allergic rhinitis. Allergen-induced nasal congestion and increased LTB₄ levels in nasal lavage fluid of patients with allergic rhinitis were significantly decreased by treatment with LTB₄ antagonists. Leukotrienes are potent inflammatory mediators of chemotaxis, bronchoconstriction, and vascular permeation, which are predominately produced by leucocytes but also by other inflammatory immune cells and in cough-associated eosinophilic inflammation, blockade of leukotriene receptors has been shown to be efficacious in cold and cough treatment.

Histamine: Histamine is an active substance that plays a major role in an allergic reaction, dilating blood vessels and increasing the permeability of vessel walls. It exerts its effects by binding to histamine receptors (H1, H2, H3, and H4) on cells' surfaces. The H1 histamine receptor plays an important role in allergic response and is widely distributed in smooth muscle, where its activation causes vasoconstriction. Activation of the H1 receptor also causes blood vessel dilation, increased vessel permeability, stimulation of sensory nerves in the airways, and bronchoconstriction. In addition, activation of this receptor promotes the chemotaxis of eosinophils, which can lead to nasal congestion, sneezing, and rhinorrhea. Hence Antihistamines are effectively used for the treatment of some allergic disorders, which act by competing with histamine released from mast cells in mucous membranes for receptor sites in target organs. Upper respiratory tract disorders are the most common causes of chronic and acute cough in humans. It has recently been proposed that the host of upper respiratory disorders that frequently cause cough to be collectively described as upper airway cough syndrome (UACS). A possible mechanism by which antihistamines could inhibit pathologic cough is by binding to nonhistaminergic receptors in the central nervous system that control cough excitability which could further regulate nasal mucus secretion.

Hence, G. glabra extract containing zinc/potassium double salt of glycyrrhizin (Gly-2), G. glabra extract containing zinc salt of glycyrrhizin (Gly-4) and G. glabra extract containing potassium salt of glycyrrhizin (Gly-5); were screened for their IFNγ and IL-2 production in comparison with regular G. glabra extract (Gly-1). Interestingly, G. glabra extract containing salts potently elevated the levels of said cytokines; and showed greater improvements in the production of IFN-γ and IL-2 compared to the regular G. glabra extract.

For example, IFNγ production activity of G. glabra extract containing zinc/potassium double salt of glycyrrhizin (Gly-2) showed an 18.18% increase in IFNγ production at 10 μg/mL, whereas G. glabra regular extract (Gly-1) showed a 13.86% increase in IFNγ production at 10 μg/mL. This is a surprising and unexpected result for enhancement of IFNγ production activity of the G. glabra extract containing zinc/potassium double salt of glycyrrhizin (Gly-2) compared to the corresponding G. glabra direct extract (Gly-1; Table-1). Similarly, G. glabra extract containing zinc salt of glycyrrhizin (Gly-4) and G. glabra extract containing potassium salt of glycyrrhizin (Gly-5) also showed higher efficacy in IFNγ production activity than the G. glabra direct extract (Gly-1).

TABLE 1
Percent increase of IFNγ and IL-2 production of the G. glabra extract
containing metal salt of glycyrrhizin and direct extract (Gly-1)

| | | % Increase | % Increase |
|---|---|---|---|
| | | of IFN-γ | of IL-2 |
| Example | | production at | production at |
| # | Product Description | 10 μg/mL | 10 μg/mL |
| 1A | G. glabra regular extract | 13.86 | 7.96 |
| | (Gly-1) | | |
| 1 | G. glabra extract containing | 18.18 | 12.93 |
| | zinc/potassium double salt | | |
| | of glycyrrhizin (Gly-2) | | |
| 2 | G. glabra extract containing | 17.2 | 10.22 |
| | zinc salt of glycyrrhizin | | |
| | (Gly-4) | | |
| 3 | G. glabra extract containing | 17.49 | 10.70 |
| | potassium salt of | | |
| | glycyrrhizin (Gly-5) | | |

Similarly, IL-2 production assay of G. glabra extract containing zinc/potassium double salt of glycyrrhizin (Gly-2) showed a 12.93% increase in IL-2 production at 10 μg/mL, whereas G. glabra regular extract (Gly-1) showed a 7.96% increase in IL-2 production at 10 μg/mL. This is also a surprising and unexpected improvement in IL-2 production activity of the G. glabra extract containing zinc/potassium double salt of glycyrrhizin (Gly-2, Table 1). Similarly, other salts also showed higher efficacy in IL-2 production activity than the G. glabra regular extract (Gly-1).

Thus the present invention provides improved Glycyrrhiza glabra extract containing at least one metal salt of glycyrrhizin selected from zinc salt of glycyrrhizin, potassium salt of glycyrrhizin, zinc/potassium double salt of glycyrrhizin; wherein Glycyrrhiza glabra extract comprises glycyrrhizin in the range of 2.0-50%, zinc in the range of 0.1-5.0% and potassium in the range of 0.1-10.0%.

### Compositions

Encouraged by the increased efficacy of Glycyrrhiza glabra extract containing metal salts of glycyrrhizin in increasing production of IFN-7 and TL-2 levels, the inventors have prepared compositions comprising Glycyrrhiza glabra extract containing metal salts of glycyrrhizin in combination with at least one ingredient selected from the extract, fraction, phytochemical or mixtures thereof derived from Tinospora cordifolia, Zingiber officinale, Justicia adhatoda, and Ocimum sanctum; to explore the efficacy of these compositions in improving immunity and respiratory health.

Thus, various solvent extracts of Tinospora cordifolia or Zingiber officinale or Justicia adhatoda, or Ocimum sanctum; were prepared. T. cordifolia dried stem was pulverized, and the powder was extracted with water, and the extract was concentrated to obtain water extract (T.C-1). Similarly, T. cordifolia dried stem powder was extracted with other solvents such as 50% aq ethanol and ethanol to obtain 50% aq ethanol extract (T.C-2) and ethanol extract (T.C-3), respectively. These extracts of T. cordifolia were standardized to 8-hydroxytinosporide by the analytical HPLC method of analysis, and the results are summarized in Table 4.

Similarly, various solvent extracts of Zingiber officinale were prepared as shown in examples 9-11, and these extracts were standardized to total gingerols and shogaols as summarized in Table 5. Similarly, various solvent extracts of Justicia adhatoda and Ocimum sanctum were also prepared, as shown in examples 13-19, and these extracts were standardized to vasicine and total of oleanolic acid and ursolic acid, respectively, as summarized in Tables 6 & 7.

The inventors then prepared four compositions; (a) composition-2 comprising G. glabra extract containing zinc/potassium double salt of glycyrrhizin (Gly-2) and T. cordifolia water extract (T.C-1) in the ratio of 2:1; (b) composition-2A comprising G. glabra extract (Gly-1) and T. cordifolia water extract (T.C-1) in the ratio of 2:1, a comparative composition without salt; (c) composition-7 comprising G. glabra extract containing zinc/potassium double salt of glycyrrhizin (Gly-2) and Z. officinale oleoresin-2 (Z.O-2) in the ratio of 3:1; and (d) composition-7A comprising G. glabra regular extract (Gly-1) and Z. officinale oleoresin-2 (Z.O-2) in the ratio of 3:1, a comparative composition without salt. These four compositions are tested for their efficacy in increasing the production of IFN-7 and L-2 in in-vitro cellular models, and the results are summarized in Table 2.

TABLE 2
Efficacy of compositions comprising Glycyrrhiza glabra extract
containing zinc/potassium salt of glycyrrhizin or regular extract and
Tinospora cordifolia water extract or Zingiber officinale oleoresin-2

| | | % Increase in | % Increase in |
|---|---|---|---|
| | | IFN-γ | IL-2 |
| Comp | | production at | production at |
| # | Composition Description | 10 μg/mL | 10 μg/mL |
| Comp- | G. glabra extract (Gly-1) and | 16.81 | 10.63 |
| 2A | T. cordifolia water extract | | |
| | (T.C-1) in the ratio of 2:1 | | |
| Comp- | G. glabra extract containing | 23.18 | 15.96 |
| 2 | zinc/potassium salt of | | |
| | glycyrrhizin (Gly-2) and T. | | |
| | cordifolia water extract (T.C- | | |
| | 1) in the ratio of 2:1 | | |
| Comp- | G. glabra extract (Gly-1) and | 18.23 | 9.94 |
| 7A | Z. officinale oleoresin-2 (Z.O- | | |
| | 2) in the ratio of 3:1 | | |
| Comp- | G. glabra extract containing | 22.87 | 15.86 |
| 7 | zinc/potassium salt of | | |
| | glycyrrhizin (Gly-2) and Z. | | |
| | officinale oleoresin-2 (Z.O-2) | | |
| | in the ratio of 3:1 | | |

From the above table, IFN-γ production of the composition-2 comprising G. glabra extract containing zinc/potassium double salt of glycyrrhizin (Gly-2) and T. cordifolia water extract (T.C-1) in the ratio of 2:1 showed 23.18% increase at 10 μg/mL. While similar composition with regular G. glabra extract without salt (comp-2A) showed a 16.81% increase in IFN-γ production at 10 μg/mL, this is a surprising and unexpected result for enhancement of IFN-γ production activity of the composition-2 compared to the corresponding comparative composition without salt (Table 2). Similarly, IL-2 production of the composition-2 showed a 15.96% increase at 10 μg/mL, whereas the similar composition without salt (comp-2A) showed a 10.63% increase in IL-2 production at 10 μg/mL. This is also a surprising and unexpected improvement. Similarly, composition-7 comprising G. glabra extract containing zinc/potassium double salt of glycyrrhizin (Gly-2) and Z. officinale oleoresin-2 (Z.O-2) in the ratio of 3:1 also showed surprisingly higher efficacy in improving IFN-γ and IL-2 production than the composition without salt (comp-7A). Hence, the compositions comprising G. glabra extract containing zinc/potassium double salt of glycyrrhizin (Gly-2) with other herbal extracts such as T. cordifolia or Z. officinale showed better efficacy in increasing production of IFN-γ and IL-2 when compared to the corresponding compositions without salt.

Following this surprise result, various compositions of Glycyrrhiza glabra extract containing metal salt of glycyrrhizin selected from zinc salt of glycyrrhizin, potassium salt of glycyrrhizin, zinc/potassium double salt of glycyrrhizin; in combination with at least one extract derived from Tinospora cordifolia, Zingiber officinale, Justicia adhatoda, and Ocimum sanctum; were prepared as summarized in examples 20-32.

Then, these compositions (C1-C44) were tested for their efficacy in increasing the production of IFN-γ, IL-2, and inhibition of LTB₄, Histamine release in in-vitro cellular models compared to the corresponding individual ingredients. Unexpectedly, these compositions showed synergistic activity.

For example, G. glabra extract containing zinc/potassium double salt of glycyrrhizin (Gly-2) at 7.5 μg/mL concentration and Tinospora cordifolia water extract (T.C-1) at 2.5 μg/mL concentration showed 13.64% and 3.01% increase in IFN-γ production, respectively. The composition-1 (C-1) containing these two ingredients (Gly-2 and T.C-1) in the ratio of 3:1 at 10 μg/mL showed a 23.49% increase in IFN-γ production, which is significantly higher than the additive effect of 16.65% (13.64%+3.01%) calculated from the increase in IFN-γ production showed by the corresponding individual ingredients (Table 8). The other compositions 2-44 (C-2 to C-44) comprising G. glabra extract containing zinc salt of glycyrrhizin or potassium salt of glycyrrhizin or zinc/potassium double salt of glycyrrhizin and one ingredient selected from the extract, fraction, phytochemical or mixtures thereof derived from Tinospora cordifolia, Zingiber officinale, Justicia adhatoda, and Ocimum sanctum; also exhibited synergism when compared to the increase in IFN-γ production shown by each of their corresponding individual ingredient concentrations as summarized in Tables 8-12.

Further, the compositions (C1-C44) also showed greater increases in IL-2 production than the corresponding individual ingredients. For example, G. glabra extract containing zinc/potassium double salt of glycyrrhizin (Gly-2) at 7.5 μg/mL concentration and T. cordifolia water extract (T.C-1) at 2.5 μg/mL concentration showed 9.70% and 2.43% increase in IL-2 production, respectively. The composition-1 (C-1) containing these two ingredients (Gly-2 and T.C-1) in the ratio of 3:1 at 10 μg/mL showed a 16.23% increase in IL-2 production, which is significantly higher than the additive effect of 12.13% (9.70%+2.43%) calculated from the increase in IL-2 production showed by the corresponding individual ingredients (Table 13). The other compositions 2-44 (C-2 to C-44) comprising G. glabra extract containing zinc salt of glycyrrhizin or potassium salt of glycyrrhizin or zinc/potassium double salt of glycyrrhizin and one ingredient selected from the extract, fraction, phytochemical or mixtures thereof derived from Tinospora cordifolia, Zingiber officinale, Justicia adhatoda, and Ocimum sanctum; also exhibited synergism when compared to the increase in IL-2 production shown by each of their corresponding individual ingredient concentrations as summarized in Tables 13-17.

Further, the compositions (C1-C44) also showed greater inhibitions of LTB₄ than the corresponding individual ingredients. For example, G. glabra extract containing zinc/potassium double salt of glycyrrhizin (Gly-2) at 7.5 μg/mL concentration and T. cordifolia water extract (T.C-1) at 2.5 μg/mL concentration showed 18.49% and 4.61% inhibition of LTB₄, respectively. The composition-1 (C-1) containing these two ingredients (Gly-2 and T.C-1) in the ratio of 3:1 at 10 μg/mL showed a 31.98% inhibition of LTB₄, which is significantly higher than the additive effect of 23.10% (18.49%+4.61%) calculated from the inhibition of LTB₄ showed by the corresponding individual ingredients (Table 18). The other compositions 2-44 (C-2 to C-44) also showed synergistic inhibition of LTB₄, as summarized in Tables 18-22.

Further, the compositions (C1-C44) also showed greater inhibition of Histamine release than the corresponding individual ingredients. For example, G. glabra extract containing zinc/potassium double salt of glycyrrhizin (Gly-2) at 7.5 μg/mL concentration and T. cordifolia water extract (T.C-1) at 2.5 μg/mL concentration showed 36.46% and 8.92% inhibition of Histamine release, respectively. The composition-1 (C-1) containing these two ingredients (Gly-2 and T.C-1) in the ratio of 3:1 at 10 μg/mL showed a 62.29% inhibition of Histamine release, which is significantly higher than the additive effect of 45.41% (36.46%+8.92%) calculated from the inhibition of Histamine release shown by the corresponding individual ingredients (Table 23). The other compositions 2-44 (C-2 to C-44) also showed synergistic inhibition of Histamine release as summarized in Tables 23-27.

Hence, these compositions (C-1 to C-44) unexpectedly showed better efficacy in increasing the production of IFN-γ and IL-2 and inhibiting LTB₄; and increasing Histamine release when compared to their corresponding individual ingredients. Thus, the compositions comprising a first ingredient Glycyrrhiza glabra extract containing metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; have the tendency to show synergism when the corresponding individual ingredients are combined.

Process: The inventors achieved two methods for the preparation of these compositions; (i) neutralization of Glycyrrhiza glabra extract with corresponding metal compound(s); (ii) purifying the extract through cation exchange resin and then conducting neutralization as summarized below in FIG. 2.

A second method, i.e., preparation of zinc/potassium double salt of glycyrrhizin, is achieved by a novel method. Purified glycyrrhizic acid is obtained by passing the 50% aqueous ethanol extract of G. glabra root through a cation exchange resin column. Glycyrrhizic acid thus obtained is neutralized with a metal oxide such as zinc oxide followed by metal carbonate such as potassium carbonate or metal hydroxide such as potassium hydroxide or mixtures thereof to yield zinc/potassium double salt of glycyrrhizin (Gly-3).

Thus, the process for the preparation of synergistic compositions comprising a first ingredient Glycyrrhiza glabra extract containing at least one metal salt of glycyrrhizin selected from zinc salt of glycyrrhizin, the potassium salt of glycyrrhizin, zinc/potassium double salt of glycyrrhizin and mixtures thereof: and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents; wherein the process comprises the following steps of,
(i) extracting dried Glycyrrhiza glabra root powder with suitable solvent;
(ii) optionally passing the extract solution from step (i) through a cation exchange resin;
(iii) neutralizing the solution of step (i) or step (ii) with metal compound(s);
(iv) filtering the solution;
(v) evaporating the solvent and drying the residue to obtain Glycyrrhiza glabra extract containing metal salt of glycyrrhizin;
(vi) blending Glycyrrhiza glabra extract containing metal salt of glycyrrhizin in step (v) with at least one extract derived from Tinospora cordifolia, Zingiber officinale, Justicia adhatoda, and Ocimum sanctum; optionally in the presence of pharmaceutically or nutraceutically or dietically acceptable excipients, carriers, and diluents; and
(vii) drying the product under a vacuum to get the compositions.

The suitable solvent used in the process for the preparation of the extracts or fractions or phytochemicals or mixtures thereof is selected from; C1-C5 alcohols selected from ethanol, methanol, n-propanol, isopropyl alcohol; ketones selected from acetone, methylisobutyl ketone; chlorinated solvents selected from methylene dichloride and chloroform; water and mixtures thereof, C1-C7 hydrocarbons such as hexane; esters like ethyl acetate and the like and mixtures thereof. The metal used in the process for the preparation of the compositions is selected from zinc and potassium, and the metal compound used for the preparation of these compositions is in the form of their metal salts, metal oxides, metal hydroxides, or carbonates.

Examples include but are not limited to zinc oxide, zinc carbonate, zinc hydroxide, potassium hydroxide, and potassium carbonate. The cation exchange resin used in the process for the preparation of the compositions is selected from strong and weak cation exchange resins, which include but not limited to Tulson T 42, Tulson CXO 12, Dowex marathon, Indion 225, Indion 236, Indion 730, Indion 652, Diaion SK1B, Diaion PK216, C 100, C 104, C 107, C 800 LT, C 800 MP, C 145, CG 10, D 001, Amberlite IRC 86, WK 11, WK 40L, D113.

Formulations: The present invention also provides synergistic herbal compositions comprising; the first ingredient Glycyrrhiza glabra extract containing at least one metal salt of glycyrrhizin selected from zinc salt of glycyrrhizin, the potassium salt of glycyrrhizin, zinc/potassium double salt of glycyrrhizin, and mixtures thereof, and a second ingredient selected from the extract, fraction, phytochemical or mixtures thereof derived from Tinospora cordifolia, Zingiber officinale, Justicia adhatoda, and Ocimum sanctum; optionally with at least one component selected from pharmaceutically or nutraceutically or dietically acceptable excipients, carriers and diluents.

The synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from the extract, fraction, phytochemical or mixtures thereof derived from Tinospora cordifolia, Zingiber officinale, Justicia adhatoda, and Ocimum sanctum; wherein the composition may be formulated with at least one component selected from pharmaceutically or nutraceutically or dietically acceptable excipients, carriers and diluents; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose; wherein the pharmaceutically or nutraceutically or dietically acceptable excipients, carriers and diluents are selected from monosaccharide's such as glucose, dextrose, fructose, galactose etc.; disaccharides such as but not limited to sucrose, maltose, lactose, lactulose, trehalose cellobiose, chitobiose etc.; polycarbohydrates such as starch and modified starch such as sodium starch glycolate, pre-gelatinized starch, soluble starch, and other modified starches; dextrins that are produced by hydrolysis of starch or glycogen such as yellow dextrin, white dextrin, maltodextrin etc.; polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide etc.; metallic stearates such as but not limited to calcium stearate, magnesium stearate, zinc stearate etc.; organic acids such as citric acid, tartaric acid, malic acid, succinic acid, lactic acid, L-ascorbic acid etc.; fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; natural pigments, flavors, class I & class II preservatives and aqueous, alcoholic, hydro-alcoholic, organic solutions of above listed ingredients alone or in combination.

In-vivo study of the compositions for airway allergic inflammation and immune modulation in ovalbumin-induced C57BL/6 mice: The compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; were evaluated for modulation of inflammation and immune response in ovalbumin-induced mice.

Allergic airway inflammation (AAI) is a serious global health concern affecting the mucosal tissue of the nasal cavity. The AAI is caused due to allergens, which are responsible for the induction of hypersensitivity reactions in the affected individuals. The allergens are any chemical or agent found indoor or outdoor, such as dust, mites, pollens, paints, molds, etc., to induce a cascade of inflammatory reactions mediated via the allergen-specific immunoglobulins. Acute activation and infiltration of inflammatory cells, which include neutrophils, lymphocytes, mast cells, eosinophils, into the bronchial fluid and in the lungs, are the most characteristic features in airway inflammation. These inflammatory cells, upon recruitment, lead to the production of various inflammatory mediators.

Ovalbumin-induced allergic airway inflammation in mice is a well-established and widely studied experimental model to study airway inflammation and immunomodulation. In ovalbumin (OVA)-induced animals, CD4+ T cells play a major role in the initiation and maintenance of T helper cell, type 2 (Th2)-like allergic airway inflammation, that is characterized by increased number of eosinophil count (eosinophilia). CD4+ T-cells play an important role in cell-mediated immune protection. CD4+ T cells produce Th2 cytokines that enhance immunoglobulin E (IgE) production and eosinophil accumulation. Therefore, a treatment that modulates the humoral immune response to suppress IgE production may be useful in reducing allergic reactions in inflammatory immune diseases, including allergic airway inflammation and other microbial infections. OVA induction causes increased accumulation of inflammatory cells that include neutrophil (neutrophilia), eosinophil in the bronchiolar lavage fluid in animals. In allergic airway inflammation, an increased level of mast cells in the lungs is an essential characteristic feature. Mast cells produce histamine. Histamine is a potent inflammatory mediator associated with allergic reactions, promoting vascular and tissue changes and promotes inflammatory cells migration. Mast cells also mediate systemic immunosuppression induced by platelet-activating factors via the histamine-dependent mechanism. Therefore, a treatment that reduces the number of mast cells can decrease the inflammatory and allergic reactions and elicit an immune response in the body. Further, the experimental evidence suggests that interferon-gamma (IFN-γ) alleviates allergic airway inflammation and elicits an immune response in the host. In the majority, IFN-γ is produced from CD4+ T cells during the adaptive immune response and is essential for controlling bacterial or viral infection and host cells' survival.

Thus the effect of T. cordifolia water extract (T.C-1), G. glabra extract containing zinc/potassium double salt of glycyrrhizin (Gly-2), and their composition (Comp-45) on some of the major biomarkers that modulate inflammation and immune responses were explored in OVA-induced C57BL/6 experimental mice. The present observations reveal that the mice supplemented with the inventive compositions showed synergistic improvements in the modulation of the biomarkers such as Total Leucocytes, Neutrophils, Lymphocytes, and Eosinophils; serum interferon-gamma (IFN-γ) and IgE; inflammatory cells and mast cells; and CD4+ T-cell subset as summarized in Tables 28-32.

Decrease of Total Leucocytes, Neutrophils, Lymphocytes, and Eosinophils: The present compositions showed synergistic efficacy in decreasing Total Leucocytes, Neutrophils, Lymphocytes, and Eosinophils in the experimental animals. For example, G. glabra extract containing zinc/potassium double salt of glycyrrhizin (Gly-2) and T. cordifolia water extract (T.C-1) supplemented animals showed 36.4% and 30.3% decreases in total Leucocytes respectively from OVA-induced mice. Comp-45 containing these two extracts at 2:1 ratio showed a 54.5% decrease from the OVA-induced mice, which is a significantly higher decrease than the corresponding individual ingredients, suggesting a synergistic effect between G. glabra extract containing zinc/potassium double salt of glycyrrhizin (Gly-2) and T. cordifolia water extract (T.C-1) in decreasing total Leucocytes (Table 28). Similarly, comp-45 also showed significantly higher decreases than the corresponding individual ingredients in the number of Neutrophils, Lymphocytes, and Eosinophils, as summarized in Tables 28 & 29.

Increase in serum interferon-gamma (IFN-γ) and decrease in IgE: The present compositions showed synergistic efficacies in modulating serum interferon-gamma (IFN-γ) and IgE in the experimental animals. For example, G. glabra extract containing zinc/potassium double salt of glycyrrhizin (Gly-2) and T. cordifolia water extract (T.C-1) supplemented animals showed 17.6% and 14.0% increase in IFN-γ respectively from OVA-induced mice. Comp-45 containing these two extracts at 2:1 ratio showed a 24.2% increase from the OVA-induced mice, which is a significantly higher increase than the corresponding individual ingredients, suggesting a synergistic effect between G. glabra extract containing zinc/potassium double salt of glycyrrhizin (Gly-2) and T. cordifolia water extract (T.C-1) in increasing IFN-γ. Similarly, comp-45 also showed a significantly higher decrease than the corresponding individual ingredients in IgE, as summarized in Table 30.

Decrease in inflammatory cells and mast cells in the lungs: The present compositions showed synergistic efficacy in decreasing of inflammatory cells and mast cells in the lungs of experimental animals. For example, G. glabra extract containing zinc/potassium double salt of glycyrrhizin (Gly-2) and T. cordifolia water extract (T.C-1) supplemented animals showed 38.7% and 29.0% decrease in total inflammation score (presence of peribroncheal+perivascular PMN cells), respectively from OVA-induced mice. Comp-45 containing these two extracts at 2:1 ratio showed a 51.6% decrease from the OVA-induced mice, which is a significantly higher decrease than the corresponding individual ingredients, suggesting a synergistic effect between G. glabra extract containing zinc/potassium double salt of glycyrrhizin (Gly-2) and T. cordifolia water extract (T.C-1) in decreasing total inflammation score. Similarly, comp-45 also showed a significantly higher decrease in mast cells than the corresponding individual ingredients, as summarized in Table 31.

Increase in CD4+ and CD8+ T-cell subsets in the spleen: The present compositions showed synergistic efficacy in increasing the CD4+ and CD8+ T-cell populations in the spleen of the experimental animals. For example, G. glabra extract containing zinc/potassium double salt of glycyrrhizin (Gly-2) and T. cordifolia water extract (T.C-1) supplemented animals showed 10.1% and 4.8% increase in CD4+ T-cell subset, respectively, from OVA-induced mice. Comp-45 containing these two extracts at 2:1 ratio showed a 17.6% increase from the OVA-induced mice, which is a significantly higher increase than the corresponding individual ingredients, suggesting a synergistic effect between G. glabra extract containing zinc/potassium double salt of glycyrrhizin (Gly-2) and T. cordifolia water extract (T.C-1) in increasing the CD4+ T-cell population in the spleen. Similarly, comp-45 also showed a significantly higher increase than the corresponding individual ingredients, as summarized in Table 32.

The foregoing demonstrates that synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof, and a second ingredient selected from the extract, fraction, phytochemical or mixtures thereof derived from Tinospora cordifolia, Zingiber officinale, Justicia adhatoda, and Ocimum sanctum; unexpectedly showed better efficacy in increasing the production of IFN-γ and IL-2, and inhibition of LTB₄ and histamine release; when compared to their corresponding individual ingredients. Hence, the said compositions can be useful for ameliorating immunity, lung function, and respiratory health.

Therefore, in an important embodiment, the present invention provides synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; for obtaining at least one health benefit selected from improving immunity/eliciting immune response/rejuvenating the immune system; improving symptoms associated with innate immunity, adaptive immunity, cellular immunity and humoral immunity; strengthen natural defense, preventing viral respiratory infections, improving lung function, supporting respiratory health, and treating/alleviating symptoms associated with airway inflammation and allergic rhinitis that include cold, cough, runny, and itchy nose.

In one preferred embodiment, the present invention provides synergistic herbal compositions comprising a first ingredient Glycyrrhiza glabra extract containing zinc salt of glycyrrhizin; and a second ingredient selected from extract, fraction, phytochemical or mixtures thereof derived from Tinospora cordifolia, Zingiber officinale, Justicia adhatoda, and Ocimum sanctum; for improving immunity, respiratory health that include allergic airway inflammation, cold and cough.

In one preferred embodiment, the present invention provides synergistic herbal compositions comprising a first ingredient Glycyrrhiza glabra extract containing potassium salt of glycyrrhizin; and a second ingredient selected from extract, fraction, phytochemical or mixtures thereof derived from Tinospora cordifolia, Zingiber officinale, Justicia adhatoda, and Ocimum sanctum; for improving immunity, respiratory health that include allergic airway inflammation, cold and cough.

In one preferred embodiment, the present invention provides synergistic herbal compositions comprising a first ingredient Glycyrrhiza glabra extract containing zinc/potassium double salt of glycyrrhizin; and a second ingredient selected from extract, fraction, phytochemical or mixtures thereof derived from Tinospora cordifolia, Zingiber officinale, Justicia adhatoda, and Ocimum sanctum; for improving immunity, respiratory health that include allergic airway inflammation, cold and cough.

In another embodiment, the present invention provides synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; wherein glycyrrhizin can be in the range of 2.0-50% and zinc can be in the range of 0.1-5.0% and potassium can be in the range of 0.1-10.0%.

In another embodiment, the present invention provides synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; wherein the concentration of Glycyrrhiza glabra extract containing metal salt in the composition varies in the range of 10%-90% by weight and the concentration of the extract of Tinospora cordifolia or Zingiber officinale or Justicia adhatoda or Ocimum sanctum; in the composition varies in the range of 90%-10% by weight.

In another embodiment, the present invention provides synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; wherein the extracts or fractions or phytochemicals or mixtures thereof are obtained from at least one plant part selected from the group comprising leaves, stems, tender stems, tender twigs, aerial parts, whole fruit, fruit peel rind, seeds, flower heads, root, bark, hardwood, rhizome or whole plant or mixtures thereof.

In another embodiment, the present invention provides synergistic herbal compositions as disclosed above; wherein the extract, fraction, phytochemical or mixtures thereof, are produced using at least one solvent selected from C1-C5 alcohols selected from ethanol, methanol, n-propanol, isopropyl alcohol; ketones selected from acetone, methylisobutyl ketone, chlorinated solvents selected from methylene dichloride and chloroform; water and mixtures thereof; C1-C7 hydrocarbons such as hexane; esters like ethyl acetate and the like and mixtures thereof.

In another embodiment, the present invention provides synergistic herbal compositions as described above; wherein the extract, fraction or mixtures thereof, in the composition are standardized to at least one phytochemical reference marker compound or pharmacologically active marker; wherein phytochemical marker compound or group of phytochemical compounds is in the concentration range of 0.01% to 90% by weight of the extract.

In another embodiment, the present invention provides synergistic herbal compositions comprising a first ingredient Glycyrrhiza glabra extract containing at least one metal salt of glycyrrhizin selected from zinc salt of glycyrrhizin, the potassium salt of glycyrrhizin, zinc/potassium double salt of glycyrrhizin, and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

In another embodiment, the present invention provides method of obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose in a human; wherein the method comprises supplementing the said human with an effective dose of a composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof, and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

In another embodiment, the present invention provides the use of a synergistic herbal composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents; for obtaining at least one health benefit selected from improving immunity/eliciting immune response/rejuvenating the immune system; improving symptoms associated with innate immunity, adaptive immunity, cellular immunity and humoral immunity; strengthen natural defense, preventing viral respiratory infections, improving lung function, supporting respiratory health, and treating/alleviating symptoms associated with airway inflammation and allergic rhinitis that include cold, cough, runny, and itchy nose.

In another embodiment of the invention, the composition as disclosed above is formulated into a dosage form selected from dry powder form, liquid form, beverage, food product, dietary supplement, or any suitable form such as a tablet, a capsule, a soft chewable tablet or gummy bear.

In another embodiment of the invention, the composition as disclosed above can be formulated into nutritional/dietary supplements that can be contemplated/made into the dosage form of healthy foods, or food for specified health uses such as solid food like chocolate or nutritional bars, semisolid food like cream, jam, or gel or beverage such as refreshing beverage, lactic acid bacteria beverage, drop, candy, chewing gum, gummy candy, yoghurt, ice cream, pudding, soft adzuki bean jelly, jelly, cookie, tea, soft drink, juice, milk, coffee, cereal, snack bar and the like.

In another embodiment of the invention, the composition as disclosed above can be formulated into controlled-release tablets, using controlled release polymer-based coatings by the techniques including nanotechnology, microencapsulation, colloidal carrier systems and other drug delivery systems for obtaining the desired therapeutic benefit.

Those of ordinary skilled in the art will appreciate that changes could be made to the embodiments described above without departing from the broad inventive concept thereof. It is understood, therefore, that this invention is not limited to the particular embodiments or examples disclosed herein, but is intended to cover modifications within the objectives and scope of the present invention as defined in the specification. The examples are given solely for the purpose of illustration and are not to be construed as limitations of the present disclosure, as many variations thereof possible without departing from the spirit of the disclosure.

### EXAMPLES

### Example 1: Glycyrrhiza glabra Extract Containing Zinc and Potassium Double Salt of Glycyrrhizin Method-1 (Gly-2)

To dried root powder of G. glabra (400 g) was added 50% aqueous ethanol (3.2 L) and the mixture was extracted at ambient temperature for 2 h. The mixture was filtered and the extraction process was repeated with 50% aqueous ethanol (2×2.4 L). The combined 50% aqueous ethanol extract was evaporated under reduced pressure up to 1.6 L of volume and the concentrate was diluted with water (1.6 L). Zinc oxide (2.16 g) and potassium carbonate were added in succession at 1 h interval, and the mixture was stirred at ambient temperature for 2 h. The mixture was filtered through celite, and the filtrate was evaporated under reduced pressure to give the product (Gly-2) as a brown color solid (114 g).

Method-2 (Gly-3): The 50% aq ethanol extract prepared from 50 g of G. glabra dried root powder as described in method 1 was passed through a strong cation exchange resin (T-42H⁺) column. The column was further eluted with 50% aqueous ethanol (400 mL). The acidic fractions were collected and mixed to give the total eluent. To the total eluent was added potassium carbonate (610 mg), zinc oxide (270 mg), potassium carbonate (300 mg), and potassium hydroxide (120 mg) in succession at 1 h intervals. The mixture was stirred at ambient temperature for 1 h, filtered, and the filtrate was evaporated under reduced pressure to give the product as a pale brown color solid (13.0 g).

### Example 1A: Glycyrrhiza glabra Extract (Gly-1)

For comparison, G. glabra extract was prepared using the following procedure: G. glabra dried root powder (50 g) was extracted with 50% aqueous ethanol as described in example 1, and evaporation of the extract gave the product as a brown color solid (13.5 g).

### Example 2: Glycyrrhiza glabra Extract Containing Zinc Salt of Glycyrrhizin (Gly-4)

To the total acidic eluent fraction obtained from 100 g of G. glabra dried root powder, as in example 1 was added zinc oxide (540 mg). The mixture was stirred at ambient temperature for 2 h, filtered, and the filtrate was evaporated under reduced pressure to give the product as a pale brown color solid (27 g).

### Example 3: Glycyrrhiza glabra Extract Containing Potassium Salt of Glycyrrhizin (Gly-5)

G. glabra extract containing potassium salt was prepared from G. glabra dried root powder (50 g) as described in example 2 by replacing zinc oxide with potassium carbonate (915 mg) or potassium hydroxide or mixtures thereof, to give the product as a pale brown color solid (13.4 g).

### Example 4: Standardization of Glycyrrhiza glabra Extracts Containing Metal Salts

The G. glabra extracts containing metal salts disclosed above were analyzed for glycyrrhizic acid by analytical HPLC. The concentration of metals such as zinc, and potassium were analyzed by ICP-MS, and the results are summarized in Table 3.

TABLE 3
Analysis data of Glycyrrhiza glabra extracts containing metal salts

| Example | | Glycyrrhizic | Metal (%) by |
|---|---|---|---|
| # | Product description | acid by HPLC | ICP-MS |
| 1 (Method- | Zinc and potassium | 14.88% | Zn: 0.9%; |
| 1) | | | K: 2.5% |
| 1 (Method- | double salt | 15.64% | Zn: 1.2%; |
| 2) | | | K: 3.2% |
| 1A | Without metal salt/s | 12.71% | — |
| 2 | Zinc salt | 14.12% | Zn: 1.4% |
| 3 | Potassium salt | 14.25% | K: 3.5% |

### Example 5: Tinospora Cordifolia Water Extract (T.C-1)

To dried stem powder of T. cordifolia (100 g) was added water (250 mL), and the mixture was extracted at ambient temperature for 3 h. The mixture was filtered, and the extraction process was repeated with water (5×250 mL) under similar conditions. The combined water extract was evaporated under reduced pressure to give the product as a brown color solid (7.5 g).

### Example 6: Tinospora Cordifolia 50% Aqueous Ethanol Extract (T.C-2)

50% Aqueous ethanol extract of T. cordifolia (100 g) was prepared as described in example 5 by replacing water with 50% aq ethanol to give the product as a brown color solid (8.3 g).

### Example 7: Tinospora Cordifolia Ethanol Extract (T.C-3)

Ethanol extract of T. cordifolia (100 g) was prepared as described in example 5 by replacing water with ethanol to give the product as a brown color solid (2.1 g).

### Example 8: Standardization of Tinospora cordifolia Extracts

The various extracts of T. cordifolia were standardized to 8-hydroxytinosporide by the analytical HPLC method, and the results are summarized in Table 4.

TABLE 4
Details of Tinospora cordifolia extracts

| Example | Solvent for | 8-hydroxytinosporide |
|---|---|---|
| # | extraction | by HPLC |
| 5 | Water | 0.25% |
| 6 | 50% aqueous ethanol | 0.40% |
| 7 | Ethanol | 0.55% |

### Example 9: Zingiber officinale Oleoresin-1 (Z.O-1)

The dried rhizome powder of Z. officinale was extracted using prior art procedures to give Z. officinale oleoresin. The extract was analyzed for total gingerols, and shogaols by HPLC method of analysis and found that the extract contains 30% of total gingerols and shogaols.

### Example 10: Zingiber officinale Oleoresin-2 (Z.O-2)

Z. officinale oleoresin obtained above is further formulated using excipients such as zinc glycinate, xantham gum, Ultrasperse-A, magnesium hydroxide, MCC and syloid in a suitable solvent to get the product, which contains 5% of total zingerols and shogaols.

### Example 11: Zingiber officinale 90% Aq Ethanol Extract (Z.O-3)

To dried rhizome powder of Z. officinale (100 g) was added 90% aqueous ethanol (700 mL), and the mixture was extracted at ambient temperature for 1 h. The mixture was filtered, and the extraction process was repeated twice with 90% aqueous ethanol (2×500 mL) under similar conditions. The combined 90% aqueous ethanol extract was evaporated under reduced pressure to give the product as a pale brown color thick paste (10.1 g).

### Example 12: Standardization of Zingiber officinale Extracts

The various extracts of Z. officinale were standardized to total gingerols and shogaols by the analytical HPLC method, and the results are summarized in Table 5.

TABLE 5
Details of Zingiber officinale extracts

| Example | Solvent for | Total of gingerols and |
|---|---|---|
| # | extraction | shogaols by HPLC |
| 9 | Prior art procedure | 30% |
| 10 | Formulation with | 5% |
| | excipients | |
| 11 | 90% aq ethanol | 17.5% |

### Example 13: Justicia Adhatoda 70% Aq Methanol Extract (J.A-1)

To dried leaf powder of Justicia adhatoda (1.0 Kg) was added 70% aqueous methanol (9.0 L), and the mixture was extracted at ambient temperature for 3 h. The mixture was filtered, and the extraction process was repeated with 70% aqueous methanol (3×7.0 L) under similar conditions. The combined 70% aqueous methanol extract was evaporated under reduced pressure to give the product as a brown color solid (100 g).

### Example 14: Justicia Adhatoda Ethanol Extract (J.A-2)

Ethanol extract of J. adhatoda (100 g) was prepared as described in example 13 by replacing 70% aq methanol with ethanol to give the product as a brown color solid (5.6 g).

### Example 15: Justicia Adhatoda Enriched Extract (J.A-3)

The dried aerial parts powder of J. adhatoda (100 g) was extracted with 70% aqueous methanol as described in example 13. The product was further enriched by acidifying with 3% aqueous citric acid solution (700 mL) and washed with chloroform to remove impurities. The acidic solution was basified with ammonia solution, and the solution was extracted with chloroform. The organic layer was evaporated to give the alkaloid fraction (1.2 g), which contains majorly vasicine.

### Example 16: Standardization of Justicia adhatoda Extracts

The various extracts of J. adhatoda were standardized to vasicine by the analytical HPLC method, and the results are summarized in Table 6.

TABLE 6
Details of of Justicia adhatoda extracts

| Example # | Solvent for extraction | Vasicine by HPLC |
|---|---|---|
| 13 | 70% aq methanol | 1.45% |
| 14 | Ethanol | 2.80% |
| 15 | Enriched extract | 95.50% |

### Example 17: Ocimum sanctum 70% Aq Methanol Extract (O.S-1)

To dried aerial parts, powder of O. sanctum (100 g) was added 70% aqueous methanol (900 mL), and the mixture was stirred at ambient temperature for 1 h. The mixture was filtered, and the extraction process was repeated with 70% aqueous methanol (2×700 mL) under similar conditions. The combined 70% aqueous methanol extract was evaporated under reduced pressure to give the product as a pale green color solid (10 g).

### Example 18: Ocimum sanctum 90% aq ethanol extract (O.S-2)

90% Aq ethanol extract of O. sanctum (100 g) was prepared as described in example 17 by replacing 70% aq methanol with 90% aq ethanol to give the product as a pale brown color solid (9.0 g).

### Example 19: Standardization of Ocimum sanctum Extracts

The various extracts of O. sanctum were standardized to total oleanolic acid and ursolic acid by the analytical HPLC method, and the results are summarized in Table 7.

TABLE 7
Details of Ocimum sanctum extracts

| | Solvent for | Total oleanolic acid and |
|---|---|---|
| Example # | extraction | ursolic acid by HPLC |
| 17 | 70% aq methanol | 1.40% |
| 18 | 90% aq ethanol | 6.56% |

### Example 20: Preparation of Various Compositions of Glycyrrhiza glabra Extract Containing Zinc/Potassium Double Salt of Glycyrrhizin (Gly-2) and Tinospora cordifolia Water Extract (T.C-1)

Composition-1 (C-1): C-1 was prepared by combining Gly-2 and T.C-1 in the ratio of 3:1.
Composition-2 (C-2): C-2 was prepared by combining Gly-2 and T.C-1 in the ratio of 2:1.
Composition-3 (C-3): C-3 was prepared by combining Gly-2 and T.C-1 in the ratio of 1:1.
Composition-4 (C-4): C-4 was prepared by combining Gly-2 and T.C-1 in the ratio of 1:2.
Composition-5 (C-5): C-5 was prepared by combining Gly-2 and T.C-1 in the ratio of 1:3.

Composition for comparison (comp-2A): The composition-2A was prepared by combining G. glabra extract (Gly-1) and T. cordifolia water extract (T.C-1) in the ratio of 2:1.

### Example 21: Preparation of Various Compositions of Glycyrrhiza glabra Extract Containing Zinc/Potassium Double Salt of Glycyrrhizin (Gly-2) and Zingiber officinale Oleoresin-2 (Z.O-2)

Composition-6 (C-6): C-6 was prepared by combining Gly-2 and Z.O-2 in the ratio of 4:1.
Composition-7 (C-7): C-7 was prepared by combining Gly-2 and Z.O-2 in the ratio of 3:1.
Composition-8 (C-8): C-8 was prepared by combining Gly-2 and Z.O-2 in the ratio of 2:1.
Composition-9 (C-9): C-9 was prepared by combining Gly-2 and Z.O-2 in the ratio of 1:1.
Composition-10 (C-10): C-10 was prepared by combining Gly-2 and Z.O-2 in the ratio of 1:2.
Composition-11 (C-11): C-11 was prepared by combining Gly-2 and Z.O-2 in the ratio of 1:3.

Composition for comparison (comp-7A): The composition-7A was prepared by combining G. glabra extract (Gly-1) and Z. officinale oleoresin-2 (Z.O-2) in the ratio of 3:1.

### Example 22: Preparation of Various Compositions of G. glabra Extract Containing Zinc/Potassium Double Salt of Glycyrrhizin (Gly-2) and T. cordifolia 50% Aq Ethanol Extract (T.C-2)

Composition-12 (C-12): C-12 was prepared by combining Gly-2 and T.C-2 in the ratio of 2:1.
Composition-13 (C-13): C-13 was prepared by combining Gly-2 and T.C-2 in the ratio of 1:1.
Composition-14 (C-14): C-14 was prepared by combining Gly-2 and T.C-2 in the ratio of 1:2.

### Example 23: Preparation of Various Compositions of G. glabra Extract Containing Zinc/Potassium Double Salt of Glycyrrhizin (Gly-2) and Z. officinale 90% Aq Ethanol Extract (Z.O-3)

Composition-15 (C-15): C-15 was prepared by combining Gly-2 and Z.O-3 in the ratio of 2:1.
Composition-16 (C-16): C-16 was prepared by combining Gly-2 and Z.O-3 in the ratio of 1:1.
Composition-17 (C-17): C-17 was prepared by combining Gly-2 and Z.O-3 in the ratio of 1:2.

### Example 24: Preparation of Various Compositions of G. glabra Extract Containing Zinc/Potassium Double Salt of Glycyrrhizin (Gly-2) and J. adhatoda 70% Aq Methanol Extract (J.A-1)

Composition-18 (C-18): C-18 was prepared by combining Gly-2 and J.A-1 in the ratio of 2:1.
Composition-19 (C-19): C-19 was prepared by combining Gly-2 and J.A-1 in the ratio of 1:1.
Composition-20 (C-20): C-20 was prepared by combining Gly-2 and J.A-1 in the ratio of 1:2.

### Example 25: Preparation of Various Compositions of G. glabra Extract Containing Zinc/Potassium Double Salt of Glycyrrhizin (Gly-2) and O. sanctum 70% Aq Methanol Extract (O.S-1)

Composition-21 (C-21): C-21 was prepared by combining Gly-2 and O.S-1 in the ratio of 2:1.
Composition-22 (C-22): C-22 was prepared by combining Gly-2 and O.S-1 in the ratio of 1:1.
Composition-23 (C-23): C-23 was prepared by combining Gly-2 and O.S-1 in the ratio of 1:2.

### Example 26: Preparation of Various Compositions of Glycyrrhiza glabra Extract Containing Zinc Salt of Glycyrrhizin (Gly-4) and Tinospora cordifolia Water Extract (T.C-1)

Composition-24 (C-24): C-24 was prepared by combining Gly-4 and T.C-1 in the ratio of 2:1.
Composition-25 (C-25): C-25 was prepared by combining Gly-4 and T.C-1 in the ratio of 1:1.
Composition-26 (C-26): C-26 was prepared by combining Gly-4 and T.C-1 in the ratio of 1:2.

### Example 27: Preparation of Various Compositions of Glycyrrhiza glabra Extract Containing Zinc Salt of Glycyrrhizin (Gly-4) and Zingiber officinale Oleoresin-2 (Z.O-2)

Composition-27 (C-27): C-27 was prepared by combining Gly-4 and Z.O-2 in the ratio of 2:1.
Composition-28 (C-28): C-28 was prepared by combining Gly-4 and Z.O-2 in the ratio of 1:1.
Composition-29 (C-29): C-29 was prepared by combining Gly-4 and Z.O-2 in the ratio of 1:2.

### Example 28: Preparation of Various Compositions of Glycyrrhiza glabra Extract Containing Zinc Salt of Glycyrrhizin (Gly-4) and Justicia adhatoda Enriched Extract (J.A-3)

Composition-30 (C-30): C-30 was prepared by combining Gly-4 and J.A-3 in the ratio of 2:1.
Composition-31 (C-31): C-31 was prepared by combining Gly-4 and J.A-3 in the ratio of 1:1.
Composition-32 (C-32): C-32 was prepared by combining Gly-4 and J.A-3 in the ratio of 1:2.

### Example 29: Preparation of Various Compositions of Glycyrrhiza glabra Extract Containing Zinc Salt of Glycyrrhizin (Gly-4) and Ocimum sanctum 90% Aq Ethanol Extract (O.S-2)

Composition-33 (C-33): C-33 was prepared by combining Gly-4 and O.S-2 in the ratio of 2:1.
Composition-34 (C-34): C-34 was prepared by combining Gly-4 and O.S-2 in the ratio of 1:1.
Composition-35 (C-35): C-35 was prepared by combining Gly-4 and O.S-2 in the ratio of 1:2.

### Example 30: Preparation of Various Compositions of Glycyrrhiza glabra Extract Containing Potassium Salt of Glycyrrhizin (Gly-5) and Tinospora cordifolia Ethanol Extract (T.C-3)

Composition-36 (C-36): C-36 was prepared by combining Gly-5 and T.C-3 in the ratio of 2:1.
Composition-37 (C-37): C-37 was prepared by combining Gly-5 and T.C-3 in the ratio of 1:1.
Composition-38 (C-38): C-38 was prepared by combining Gly-5 and T.C-3 in the ratio of 1:2.

### Example 31: Preparation of Various Compositions of Glycyrrhiza glabra Extract Containing Potassium Salt of Glycyrrhizin (Gly-5) and Zingiber officinale Oleoresin-1 (Z.O-1)

Composition-39 (C-39): C-39 was prepared by combining Gly-5 and Z.O-1 in the ratio of 2:1.
Composition-40 (C-40): C-40 was prepared by combining Gly-5 and Z.O-1 in the ratio of 1:1.
Composition-41 (C-41): C-41 was prepared by combining Gly-5 and Z.O-1 in the ratio of 1:2.

### Example 32: Preparation of Various Compositions of Glycyrrhiza glabra Extract Containing Potassium Salt of Glycyrrhizin (Gly-5) and Justicia adhatoda Ethanol Extract (J.A-2)

Composition-42 (C-42): C-42 was prepared by combining Gly-5 and J.A-2 in the ratio of 2:1.
Composition-43 (C-43): C-43 was prepared by combining Gly-5 and J.A-2 in the ratio of 1:1.
Composition-44 (C-44): C-44 was prepared by combining Gly-5 and J.A-2 in the ratio of 1:2.

### Example 33: Formulation of the Compositions

Composition-45 (C-45): A mixture of G. glabra extract containing zinc/potassium double salt of glycyrrhizin (Gly-2, 63.33 g), T. cordifolia water extract (T.C-1, 31.66 g), Glucidex12D (3.0 g), and Syloid (2.0 g) was taken in a double poly bag and blend manually. The powder was pulverized and sieved through 40 mesh to give the composition as a free-flowing fine powder (Comp-45).
Composition-46 (C-46): A mixture of G. glabra extract containing zinc/potassium double salt of glycyrrhizin (Gly-2, 78.4 g), Z. officinale oleoresin-2 (Z.O-2, 19.6 g), and Syloid (2.0 g) was taken in a double poly bag and blend manually. The powder was pulverized and sieved through 40 mesh to give the composition as a free-flowing fine powder (Comp-46).

### Example 34: Assay for Interferon-7 (IFN-γ) Production

Human blood was collected from healthy volunteers from a peripheral vein with a syringe containing EDTA at a final concentration of 2 mM. Plasma was separated by centrifugation at 150×g for 10 min, and the residual blood was diluted with RPMI medium supplemented with 10% FBS and 2 mM EDTA at a ratio of 1:3. Thirty milliliters of blood were carefully layered onto 15 mL of Ficoll/Lymphoprep in a 50 mL falcon tube in the dark, and tubes were centrifuged at 350×g for 30 min. The buffy coat (interface between medium and Ficoll) containing peripheral blood mononuclear cells (PBMC) was collected carefully in 25 mL of cold 1× phosphate-buffered saline (PBS) and centrifuged at 1200 rpm for 10 min. Residual RBCs found in PBMCs pellet were removed by treating with ACK lysis buffer (Gibco, USA) and washed with fresh 1×PBS. PBMC were seeded in a 96-well plate with a density of 0.1×10⁶ cells/well and treated with different concentrations of test samples. Cells with 0.2% DMSO served as vehicle control. The plate was incubated in a CO₂ incubator at 37° C. for 2 hrs. Finally, cells were induced with the combination of Phorbol-12-myristate-13-acetate (PMA, 7.5 nM) and Phytohemagglutinin-A (PHA, 2 μg/ml) for 4 hrs except for vehicle control by keeping the plate at 37° C. in a CO₂ incubator. The plate was centrifuged at 270×g for 5 min, and 120 μL cell-free supernatants were collected. Quantification of IFN-7 was performed using the ELISA kit (R&D Systems, USA) according to the manufacturer's instructions. Absorbance was measured at 450 nm in a microplate reader (Spectramax 2e, Molecular Devices, USA). Percent increase in IFN-γ production was calculated using the following formula.

$$\% increase in {IFN}_{- γ} = \frac{( {IFN}_{γ} conc . in Test Sample ) - ( {IFN}_{γ} conc . in Induction )}{( {IFN}_{γ} conc . in Induction )} \times 100$$

The results are presented in Tables: 8-12.

TABLE 8
Percent increase in IFN-γ production by the compositions of
Glycyrrhiza glabra extract containing zinc/potassium double salt of
glycyrrhizin (Gly-2) and Tinospora cordifolia water extract (T.C-1)

| | Gly-2 | | T.C-1 | | | | % Increase in IFN-γ production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-1 | 7.5 | 13.64 | 2.5 | 3.01 | 3:1 | 10 | 16.65 | 23.49 |
| C-2 | 6.67 | 12.13 | 3.33 | 4.01 | 2:1 | 10 | 16.14 | 23.18 |
| C-3 | 5.0 | 9.09 | 5.0 | 6.02 | 1:1 | 10 | 15.11 | 21.09 |
| C-4 | 3.33 | 6.05 | 6.67 | 8.03 | 1:2 | 10 | 14.08 | 21.7 |
| C-5 | 2.5 | 4.55 | 7.5 | 9.03 | 3:1 | 10 | 13.58 | 20.23 |

TABLE 9
Percent increase in IFN-γ production by the compositions of
Glycyrrhiza glabra extract containing zinc/potassium double salt
of glycyrrhizin (Gly-2) and Zingiber officinale oleoresin-2 (Z.O-2)

| | Gly-2 | | Z.O-2 | | | | % Increase in IFN-γ production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-6 | 8.0 | 14.54 | 2.0 | 3.00 | 4:1 | 10 | 17.54 | 22.69 |
| C-7 | 7.5 | 13.64 | 2.5 | 3.75 | 3:1 | 10 | 17.38 | 22.87 |
| C-8 | 6.67 | 12.13 | 3.33 | 4.99 | 2:1 | 10 | 17.12 | 23.98 |
| C-9 | 5.0 | 9.09 | 5.0 | 7.50 | 1:1 | 10 | 16.59 | 22.75 |
| C-10 | 3.33 | 6.05 | 6.67 | 10.00 | 1:2 | 10 | 16.05 | 21.33 |
| C-11 | 2.5 | 4.55 | 7.5 | 11.24 | 3:1 | 10 | 15.79 | 22.53 |

TABLE 10
Percent increase in IFN-γ production by the compositions of Glycyrrhiza glabra extract
containing zinc/potassium double salt of glycyrrhizin (Gly-2) and T. cordifolia 50% aq
ethanol extract (T.C-2) or Z. officinale 90% aq ethanol extract (Z.O-3) or J. adhatoda
70% aq methanol extract (J.A-1) or O. sanctum 70% aq methanol extract (O.S-1)

| | Gly-2 | | T.C-2 | | | | % Increase in IFN-γ production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-12 | 6.67 | 12.13 | 3.33 | 3.05 | 2:1 | 10 | 15.18 | 19.34 |
| C-14 | 3.33 | 6.05 | 6.67 | 6.11 | 1:2 | 10 | 12.16 | 18.58 |
| | Gly-2 | | Z.O-3 | | | | | |
| C-15 | 6.67 | 12.13 | 3.33 | 4.52 | 2:1 | 10 | 16.64 | 21.5 |
| C-17 | 3.33 | 6.05 | 6.67 | 9.05 | 1:2 | 10 | 15.11 | 19.45 |
| | Gly-2 | | J.A-1 | | | | | |
| C-18 | 6.67 | 12.13 | 3.33 | 2.79 | 2:1 | 10 | 14.92 | 19.29 |
| C-20 | 3.33 | 6.05 | 6.67 | 5.60 | 1:2 | 10 | 11.65 | 15.23 |
| | Gly-2 | | O.S-1 | | | | | |
| C-21 | 6.67 | 12.13 | 3.33 | 2.40 | 2:1 | 10 | 14.53 | 18.15 |
| C-23 | 3.33 | 6.05 | 6.67 | 4.82 | 1:2 | 10 | 10.87 | 14.51 |

TABLE 11
Percent increase in IFN-γ production by the compositions of Glycyrrhiza glabra
extract containing zinc salt of glycyrrhizin (Gly-4) and T. cordifolia water extract
(T.C-1) or Z. officinale oleoresin-2 (Z.O-2) or J. adhatoda enriched extract
(J.A-3) or O. sanctum 90% aq ethanol extract (O.S-2)

| | Gly-4 | | T.C-1 | | | | % Increase in IFN-γ production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-24 | 6.67 | 11.47 | 3.33 | 4.01 | 2:1 | 10 | 15.48 | 19.96 |
| C-26 | 3.33 | 5.73 | 6.67 | 8.03 | 1:2 | 10 | 13.76 | 18.77 |
| | Gly-4 | | Z.O-2 | | | | | |
| C-27 | 6.67 | 11.47 | 3.33 | 4.99 | 2:1 | 10 | 16.46 | 21.23 |
| C-29 | 3.33 | 5.73 | 6.67 | 10.00 | 1:2 | 10 | 15.73 | 19.35 |
| | Gly-4 | | J.A-3 | | | | | |
| C-30 | 6.67 | 11.47 | 3.33 | 2.87 | 2:1 | 10 | 14.34 | 17.85 |
| C-32 | 3.33 | 5.73 | 6.67 | 5.75 | 1:2 | 10 | 11.48 | 15.09 |
| | Gly-4 | | O.S-2 | | | | | |
| C-33 | 6.67 | 11.47 | 3.33 | 3.49 | 2:1 | 10 | 14.97 | 19.19 |
| C-35 | 3.33 | 5.73 | 6.67 | 7.00 | 1:2 | 10 | 12.72 | 18.58 |

TABLE 12
Percent increase in IFN-γ production by the compositions of Glycyrrhiza glabra extract
containing potassium salt of glycyrrhizin (Gly-5) and T. cordifolia ethanol extract (T.C-3)
or Z. officinale oleoresin-1 (Z.O-1) or J. adhatoda ethanol extract (J.A-2)

| | Gly-5 | | T.C-3 | | | | % Increase in IFN-γ production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-36 | 6.67 | 11.67 | 3.33 | 2.82 | 2:1 | 10 | 14.49 | 18.15 |
| C-38 | 3.33 | 5.82 | 6.67 | 5.65 | 1:2 | 10 | 11.47 | 15.64 |
| | Gly-5 | | Z.O-1 | | | | | |
| C-39 | 6.67 | 11.67 | 3.33 | 4.32 | 2:1 | 10 | 15.98 | 20.75 |
| C-41 | 3.33 | 5.82 | 6.67 | 8.65 | 1:2 | 10 | 14.48 | 18.53 |
| | Gly-5 | | J.A-2 | | | | | |
| C-42 | 6.67 | 11.67 | 3.33 | 2.89 | 2:1 | 10 | 14.56 | 18.14 |
| C-44 | 3.33 | 5.82 | 6.67 | 5.79 | 1:2 | 10 | 11.61 | 15.33 |

### Example 35: Assay for Interleukin-2 (IL-2) Production

An equal number of Jurkat cells (0.1×10⁶) suspended in 200 μL of RPMI medium supplemented with 10% FBS was seeded in each well of a 96-well plate. Cells were pretreated with different concentrations of test samples. Cells with 0.2% DMSO served as vehicle control. The plate was incubated in a CO₂ incubator at 37° C. for 2 hrs. After the incubation period, the cells were induced with the combination of Phorbol-12-myristate-13-acetate (PMA, 7.5 nM) and Phytohemagglutinin-A (PHA, 0.5 μg/ml) for 4 hrs except for vehicle control by keeping the plate at 37° C. in a CO₂ incubator. The plate was centrifuged at 270×g for 5 min to collect the cell-free culture supernatants. Quantitation of IL-2 was performed using the ELISA kit (R&D systems Cat #DY202) according to the manufacturer's instructions. Absorbance was measured at 450 nm in a microplate reader (Spectramax 2e, Molecular Devices, USA). Percent increase in IL-2 production was calculated using the following formula.

$$\% increase in IL - 2 = \frac{( Conc . of IL - 2 in Test Sample ) - ( Conc . of IL - 2 in Induction )}{( Conc . of IL - 2 in Induction )} \times 100$$

The results are presented in Tables: 13-17.

TABLE 13
Percent increase in IL-2 production by the compositions of Glycyrrhiza glabra
extract containing zinc/potassium double salt of glycyrrhizin (Gly-2)
and Tinospora cordifolia water extract (T.C-1)

| | Gly-2 | | T.C-1 | | | | % Increase in IL-2 production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-1 | 7.5 | 9.70 | 2.5 | 2.43 | 3:1 | 10 | 12.13 | 16.23 |
| C-2 | 6.67 | 8.62 | 3.33 | 3.24 | 2:1 | 10 | 11.86 | 15.96 |
| C-3 | 5.0 | 6.47 | 5.0 | 4.86 | 1:1 | 10 | 11.33 | 14.57 |
| C-4 | 3.33 | 4.31 | 6.67 | 6.48 | 1:2 | 10 | 10.79 | 13.65 |
| C-5 | 2.5 | 3.23 | 7.5 | 7.29 | 3:1 | 10 | 10.52 | 14.37 |

TABLE 14
Percent increase in IL-2 production by the compositions of Glycyrrhiza glabra
extract containing zinc/potassium double salt of glycyrrhizin (Gly-
2) and Zingiber officinale oleoresin-2 (Z.O-2)

| | Gly-2 | | Z.O-2 | | | | % Increase in IL-2 production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-6 | 8.0 | 10.34 | 2.0 | 1.66 | 4:1 | 10 | 12.00 | 16.34 |
| C-7 | 7.5 | 9.70 | 2.5 | 2.07 | 3:1 | 10 | 11.77 | 15.86 |
| C-8 | 6.67 | 8.62 | 3.33 | 2.76 | 2:1 | 10 | 11.38 | 14.56 |
| C-9 | 5.0 | 6.47 | 5.0 | 4.15 | 1:1 | 10 | 10.61 | 15.05 |
| C-10 | 3.33 | 4.31 | 6.67 | 5.53 | 1:2 | 10 | 9.84 | 14.03 |
| C-11 | 2.5 | 3.23 | 7.5 | 6.22 | 3:1 | 10 | 9.45 | 13.07 |

TABLE 15
Percent increase in IL-2 production by the compositions of Glycyrrhiza glabra extract
containing zinc/potassium double salt of glycyrrhizin (Gly-2) and T. cordifolia 50% aq
ethanol extract (T.C-2) or Z. officinale 90% aq ethanol extract (Z.O-3) or J. adhatoda
70% aq methanol extract (J.A-1) or O. sanctum 70% aq methanol extract (O.S-1)

| | Gly-2 | | T.C-2 | | | | % Increase in IL-2 production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-12 | 6.67 | 8.62 | 3.33 | 2.02 | 2:1 | 10 | 10.65 | 13.43 |
| C-14 | 3.33 | 4.31 | 6.67 | 4.05 | 1:2 | 10 | 8.35 | 10.45 |
| | Gly-2 | | Z.O-3 | | | | | |
| C-15 | 6.67 | 8.62 | 3.33 | 2.21 | 2:1 | 10 | 10.84 | 13.63 |
| C-17 | 3.33 | 4.31 | 6.67 | 4.44 | 1:2 | 10 | 8.74 | 11.73 |
| | Gly-2 | | J.A-1 | | | | | |
| C-18 | 6.67 | 8.62 | 3.33 | 2.82 | 2:1 | 10 | 11.44 | 14.35 |
| C-20 | 3.33 | 4.31 | 6.67 | 5.64 | 1:2 | 10 | 9.95 | 12.89 |
| | Gly-2 | | O.S-1 | | | | | |
| C-21 | 6.67 | 8.62 | 3.33 | 2.67 | 2:1 | 10 | 11.29 | 14.08 |
| C-23 | 3.33 | 4.31 | 6.67 | 5.34 | 1:2 | 10 | 9.65 | 13.26 |

TABLE 16
Percent increase in IL-2 production by the compositions of Glycyrrhiza glabra
extract containing zinc salt of glycyrrhizin (Gly-4) and T. cordifolia water extract
(T.C-1) or Z. officinale oleoresin-2 (Z.O-2) or J. adhatoda enriched extract
(J.A-3) or O. sanctum 90% aq ethanol extract (O.S-2)

| | Gly-4 | | T.C-1 | | | | % Increase in IL-2 production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-24 | 6.67 | 6.82 | 3.33 | 3.24 | 2:1 | 10 | 10.05 | 12.91 |
| C-26 | 3.33 | 3.40 | 6.67 | 6.48 | 1:2 | 10 | 9.89 | 13.25 |
| | Gly-4 | | Z.O-2 | | | | | |
| C-27 | 6.67 | 6.82 | 3.33 | 2.76 | 2:1 | 10 | 9.58 | 12.55 |
| C-29 | 3.33 | 3.40 | 6.67 | 5.53 | 1:2 | 10 | 8.93 | 12.18 |
| | Gly-4 | | J.A-3 | | | | | |
| C-30 | 6.67 | 6.82 | 3.33 | 2.99 | 2:1 | 10 | 9.80 | 13.17 |
| C-32 | 3.33 | 3.40 | 6.67 | 5.98 | 1:2 | 10 | 9.39 | 12.10 |
| | Gly-4 | | O.S-2 | | | | | |
| C-33 | 6.67 | 6.82 | 3.33 | 3.14 | 2:1 | 10 | 9.96 | 12.49 |
| C-35 | 3.33 | 3.40 | 6.67 | 6.30 | 1:2 | 10 | 9.70 | 12.26 |

TABLE 17
Percent increase in IL-2 production by the compositions of Glycyrrhiza glabra extract
containing potassium salt of glycyrrhizin (Gly-5) and T. cordifolia ethanol extract (T.C-3)
or Z. officinale oleoresin-1 (Z.O-1) or J. adhatoda ethanol extract (J.A-2)

| | Gly-5 | | T.C-3 | | | | % Increase in IL-2 production | |
|---|---|---|---|---|---|---|---|---|
| Comp | | % | | % | | Dose | Additive | |
| # | μg/mL | increase | μg/mL | increase | Ratio | μg/mL | (Calculated) | Observed |
| C-36 | 6.67 | 7.14 | 3.33 | 2.45 | 2:1 | 10 | 9.58 | 12.17 |
| C-38 | 3.33 | 3.56 | 6.67 | 4.90 | 1:2 | 10 | 8.47 | 10.82 |
| | Gly-5 | | Z.O-1 | | | | | |
| C-39 | 6.67 | 7.14 | 3.33 | 2.76 | 2:1 | 10 | 9.90 | 12.48 |
| C-41 | 3.33 | 3.56 | 6.67 | 5.54 | 1:2 | 10 | 9.10 | 11.73 |
| | Gly-5 | | J.A-2 | | | | | |
| C-42 | 6.67 | 7.14 | 3.33 | 3.23 | 2:1 | 10 | 10.36 | 13.63 |
| C-44 | 3.33 | 3.56 | 6.67 | 6.46 | 1:2 | 10 | 10.03 | 13.01 |

Example 36: Assay for Leukotriene B₄ (LTB₄) inhibition Human blood was collected from healthy volunteers from a peripheral vein with a syringe containing EDTA at a final concentration of 2 mM. Plasma was separated by centrifugation at 1000 rpm for 10 min, and the residual blood was diluted with RPMI medium supplemented with 10% FBS and 2 mM EDTA at a ratio of 1:3. Thirty milliliters of blood was carefully layered onto 15 mL of Ficoll/Lyymphoprep in a 50 mL falcon tube in the dark, and the tubes were centrifuged at 350×g for 30 min. After removing the peripheral blood mononuclear cells (PBMC) and Ficoll/Lymphoprep, the settled RBC layer containing granulocytes was treated with ACK lysis buffer (Gibco Cat #A10492-01) to lyse the RBC completely. After centrifugation at 150×g for 10 min, the resulting cell pellet of polymorphonuclear leukocytes (PMNs) was resuspended in RPMI containing 1% (v/v) newborn calf serum (NBCS). An equal number of PMNs (50,000 cells) was seeded in each well of a 96-well plate and treated with different concentrations of the test samples. Cells with 0.2% DMSO were served as vehicle control. The plate was incubated in a CO₂ incubator at 37° C. for 2 hrs. Finally, cells were induced with 10 μM A23187 (Sigma Chemical Co, USA) for 10 min, except the vehicle control cells. The plate was centrifuged at 150×g for 5 min, and 120 μL of cell-free supernatants were collected. Quantitation of LTB₄ in the cell culture supernatants was performed using an LTB₄ ELISA kit (R&D Systems, USA) according to the manufacturer's instructions. Absorbance was measured at 450 nm with a correction wavelength of 570 nm in a microplate reader (Spectramax 2e, Molecular Devices, USA). Percent reduction in LTB₄ production was calculated using the following formula.

$$\% reduction in {LTB}_{4} = \frac{( Conc . of {LTB}_{4} in Induction ) - ( Conc . of {LTB}_{4} in Test Sample )}{( Conc . of {LTB}_{4} in Induction )} \times 100$$

The results are presented in Tables: 18-22.

TABLE 18
Percent inhibition in LTB₄ production by the compositions of Glycyrrhiza glabra
extract containing zinc/potassium double salt of glycyrrhizin (Gly-2)
and Tinospora cordifolia water extract (T.C-1)

| | Gly-2 | | T.C-1 | | | | % Inhibition in LTB₄ | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-1 | 7.5 | 18.49 | 2.5 | 4.61 | 3:1 | 10 | 23.10 | 31.98 |
| C-2 | 6.67 | 16.44 | 3.33 | 6.14 | 2:1 | 10 | 22.58 | 32.87 |
| C-3 | 5.0 | 12.33 | 5.0 | 9.22 | 1:1 | 10 | 21.55 | 33.09 |
| C-4 | 3.33 | 8.21 | 6.67 | 12.30 | 1:2 | 10 | 20.51 | 31.62 |
| C-5 | 2.5 | 6.16 | 7.5 | 13.83 | 3:1 | 10 | 19.99 | 29.45 |

TABLE 19
Percent inhibition in LTB₄ production by the compositions of Glycyrrhiza glabra
extract containing zinc/potassium double salt of glycyrrhizin (Gly-
2) and Zingiber officinale oleoresin-2 (Z.O-2)

| | Gly-2 | | Z.O-2 | | | | % Inhibition in LTB₄ | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-6 | 8.0 | 19.72 | 2.0 | 4.11 | 4:1 | 10 | 23.83 | 32.3 |
| C-7 | 7.5 | 18.49 | 2.5 | 5.14 | 3:1 | 10 | 23.62 | 31.1 |
| C-8 | 6.67 | 16.44 | 3.33 | 6.84 | 2:1 | 10 | 23.28 | 29.88 |
| C-9 | 5.0 | 12.33 | 5.0 | 10.27 | 1:1 | 10 | 22.60 | 31.36 |
| C-10 | 3.33 | 8.21 | 6.67 | 13.70 | 1:2 | 10 | 21.91 | 28.31 |
| C-11 | 2.5 | 6.16 | 7.5 | 15.41 | 3:1 | 10 | 21.57 | 29.16 |

TABLE 20
Percent inhibition in LTB₄ production by the compositions of Glycyrrhiza glabra extract
containing zinc/potassium double salt of glycyrrhizin (Gly-2) and T. cordifolia 50% aq ethanol
extract (T.C-2) or Z. officinale 90% aq ethanol extract (Z.O-3) or J. adhatoda 70% aq
methanol extract (J.A-1) or O. sanctum 70% aq methanol extract (O.S-1)

| | Gly-2 | | T.C-2 | | | | % Inhibition in LTB₄ | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-12 | 6.67 | 16.44 | 3.33 | 4.33 | 2:1 | 10 | 20.77 | 27.48 |
| C-14 | 3.33 | 8.21 | 6.67 | 8.68 | 1:2 | 10 | 16.89 | 23.52 |
| | Gly-2 | | Z.O-3 | | | | | |
| C-15 | 6.67 | 16.44 | 3.33 | 6.15 | 2:1 | 10 | 22.59 | 29.43 |
| C-17 | 3.33 | 8.21 | 6.67 | 12.31 | 1:2 | 10 | 20.52 | 29.86 |
| | Gly-2 | | J.A-1 | | | | | |
| C-18 | 6.67 | 16.44 | 3.33 | 6.19 | 2:1 | 10 | 22.63 | 28.37 |
| C-20 | 3.33 | 8.21 | 6.67 | 12.40 | 1:2 | 10 | 20.61 | 26.91 |
| | Gly-2 | | O.S-1 | | | | | |
| C-21 | 6.67 | 16.44 | 3.33 | 5.53 | 2:1 | 10 | 21.97 | 27.91 |
| C-23 | 3.33 | 8.21 | 6.67 | 11.07 | 1:2 | 10 | 19.28 | 26.52 |

TABLE 21
Percent inhibition in LTB₄ production by the compositions of Glycyrrhiza glabra
extract containing zinc salt of glycyrrhizin (Gly-4) and T. cordifolia water extract
(T.C-1) or Z. officinale oleoresin-2 (Z.O-2) or J. adhatoda enriched extract
(J.A-3) or O. sanctum 90% aq ethanol extract (O.S-2)

| | Gly-4 | | T.C-1 | | | | % Inhibition in LTB₄ | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-24 | 6.67 | 14.88 | 3.33 | 6.14 | 2:1 | 10 | 21.02 | 26.05 |
| C-26 | 3.33 | 7.43 | 6.67 | 12.30 | 1:2 | 10 | 19.73 | 24.9 |
| | Gly-4 | | Z.O-2 | | | | | |
| C-27 | 6.67 | 14.88 | 3.33 | 6.84 | 2:1 | 10 | 21.72 | 28.37 |
| C-29 | 3.33 | 7.43 | 6.67 | 13.70 | 1:2 | 10 | 21.13 | 25.84 |
| | Gly-4 | | J.A-3 | | | | | |
| C-30 | 6.67 | 14.88 | 3.33 | 6.19 | 2:1 | 10 | 21.07 | 26.78 |
| C-32 | 3.33 | 7.43 | 6.67 | 12.40 | 1:2 | 10 | 19.83 | 24.83 |
| | Gly-4 | | O.S-2 | | | | | |
| C-33 | 6.67 | 14.88 | 3.33 | 4.80 | 2:1 | 10 | 19.68 | 24.82 |
| C-35 | 3.33 | 7.43 | 6.67 | 9.62 | 1:2 | 10 | 17.05 | 24.32 |

TABLE 22
Percent inhibition in LTB₄ production by the compositions of Glycyrrhiza glabra extract
containing potassium salt of glycyrrhizin (Gly-5) and T. cordifolia ethanol extract (T.C-3)
or Z. officinale oleoresin-1 (Z.O-1) or J. adhatoda ethanol extract (J.A-2)

| | Gly-5 | | T.C-3 | | | | % Inhibition in LTB₄ | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-36 | 6.67 | 14.04 | 3.33 | 5.01 | 2:1 | 10 | 19.05 | 24.5 |
| C-38 | 3.33 | 7.01 | 6.67 | 10.03 | 1:2 | 10 | 17.04 | 22.42 |
| | Gly-5 | | Z.O-1 | | | | | |
| C-39 | 6.67 | 14.04 | 3.33 | 5.45 | 2:1 | 10 | 19.49 | 24.59 |
| C-41 | 3.33 | 7.01 | 6.67 | 10.92 | 1:2 | 10 | 17.93 | 22.94 |
| | Gly-5 | | J.A-2 | | | | | |
| C-42 | 6.67 | 14.04 | 3.33 | 4.33 | 2:1 | 10 | 18.37 | 22.89 |
| C-44 | 3.33 | 7.01 | 6.67 | 8.68 | 1:2 | 10 | 15.69 | 19.77 |

### Example 37: Assay for Histamine Release Inhibition

Primary rat peritoneal cells (RPCs) were utilized for the histamine release assay. Fifty milliliters of sterile Tyrode buffer was injected into the peritoneal cavity of an anesthetized rat, and the buffer containing RPCs was recovered using a sterile Pasteur pipette. The recovered RPCs were passed through a cell strainer to remove residual tissue, washed with Tyrode buffer, and centrifuged at 400×g for 10 min at 4° C. In a 96-well plate, RPCs (0.1×10⁶ cells/well) were seeded. The cells were pretreated with different concentrations of test samples and incubated at 37° C. in a CO₂ incubator for 2 hr. Next, cells were induced with 2.5 μM of A23187 (Sigma Chemical Co, USA) at 37° C. in a CO₂ incubator for 15 min. Cells with 0.2% DMSO served as vehicle control. The plate was centrifuged at 270×g for 5 min to collect the cell-free culture supernatants. Twenty microliters of 1N NaOH were added to all wells of a 96-well black clear bottom plate. To this plate, 100 μl of the cell-free supernatants were added, followed by 5 μl of O-Phthalaldehyde (OPA), and incubated for 3 min on a shaker in the dark. The reaction was stopped with 10 μl of stop solution (3N HCl) and mixed the content thoroughly. Relative fluorescence (RFU) was measured at Ex/Em: 360/440 nm in a microplate reader (Spectramax 2e, Molecular Devices, USA) for estimating the spontaneous histamine release. Percent reduction of spontaneous histamine release from the primary peritoneal cells was calculated using the following formula.

$$\% reduction in histamine release = \frac{( RFU in inducer ) - ( RFU in test samples )}{( RFU in inducer )} \times 100$$

The results are presented in Tables: 23-27.

TABLE 23
Percent inhibition in histamine release by the compositions of Glycyrrhiza glabra
extract containing zinc/potassium double salt of glycyrrhizin (Gly-2)
and Tinospora cordifolia water extract (T.C-1)

| | Gly-2 | | T.C-1 | | | | % Inhibition in histamine | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-1 | 7.5 | 36.48 | 2.5 | 8.92 | 3:1 | 10 | 45.41 | 62.29 |
| C-2 | 6.67 | 32.44 | 3.33 | 11.89 | 2:1 | 10 | 44.33 | 62.61 |
| C-3 | 5.0 | 24.32 | 5.0 | 17.85 | 1:1 | 10 | 42.17 | 63.19 |
| C-4 | 3.33 | 16.20 | 6.67 | 23.81 | 1:2 | 10 | 40.01 | 59.23 |
| C-5 | 2.5 | 12.16 | 7.5 | 26.77 | 3:1 | 10 | 38.93 | 56.82 |

TABLE 24
Percent inhibition in histamine release by the compositions of Glycyrrhiza glabra
extract containing zinc/potassium double salt of glycyrrhizin (Gly-2)
and Zingiber officinale oleoresin-2 (Z.O-2)

| | Gly-2 | | Z.O-2 | | | | % Inhibition in histamine | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-6 | 8.0 | 38.91 | 2.0 | 6.61 | 4:1 | 10 | 45.53 | 62.34 |
| C-7 | 7.5 | 36.48 | 2.5 | 8.27 | 3:1 | 10 | 44.75 | 61.76 |
| C-8 | 6.67 | 32.44 | 3.33 | 11.01 | 2:1 | 10 | 43.46 | 60.35 |
| C-9 | 5.0 | 24.32 | 5.0 | 16.54 | 1:1 | 10 | 40.86 | 61.39 |
| C-10 | 3.33 | 16.20 | 6.67 | 22.06 | 1:2 | 10 | 38.26 | 57.92 |
| C-11 | 2.5 | 12.16 | 7.5 | 24.80 | 3:1 | 10 | 36.96 | 56.16 |

TABLE 25
Percent inhibition in histamine release by the compositions of Glycyrrhiza glabra extract
containing zinc/potassium double salt of glycyrrhizin (Gly-2) and T. cordifolia 50% aq ethanol
extract (T.C-2) or Z. officinale 90% aq ethanol extract (Z.O-3) or J. adhatoda 70% aq
methanol extract (J.A-1) or O. sanctum 70% aq methanol extract (O.S-1)

| | Gly-2 | | T.C-2 | | | | % Inhibition in histamine | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-12 | 6.67 | 32.44 | 3.33 | 9.71 | 2:1 | 10 | 42.15 | 57.08 |
| C-14 | 3.33 | 16.20 | 6.67 | 19.45 | 1:2 | 10 | 35.64 | 48.54 |
| | Gly-2 | | Z.O-3 | | | | | |
| C-15 | 6.67 | 32.44 | 3.33 | 10.12 | 2:1 | 10 | 42.56 | 55.30 |
| C-17 | 3.33 | 16.20 | 6.67 | 20.26 | 1:2 | 10 | 36.46 | 53.79 |
| | Gly-2 | | J.A-1 | | | | | |
| C-18 | 6.67 | 32.44 | 3.33 | 10.60 | 2:1 | 10 | 43.04 | 54.89 |
| C-20 | 3.33 | 16.20 | 6.67 | 21.22 | 1:2 | 10 | 37.42 | 50.95 |
| | Gly-2 | | O.S-1 | | | | | |
| C-21 | 6.67 | 32.44 | 3.33 | 10.35 | 2:1 | 10 | 42.79 | 53.16 |
| C-23 | 3.33 | 16.20 | 6.67 | 20.73 | 1:2 | 10 | 36.93 | 51.93 |

TABLE 26
Percent inhibition in histamine release by the compositions of Glycyrrhiza glabra
extract containing zinc salt of glycyrrhizin (Gly-4) and T. cordifolia water extract
(T.C-1) or Z. officinale oleoresin-2 (Z.O-2) or J. adhatoda enriched extract
(J.A-3) or O. sanctum 90% aq ethanol extract (O.S-2)

| | Gly-4 | | T.C-1 | | | | % Inhibition in histamine | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-24 | 6.67 | 28.93 | 3.33 | 11.89 | 2:1 | 10 | 40.82 | 53.10 |
| C-26 | 3.33 | 14.44 | 6.67 | 23.81 | 1:2 | 10 | 38.25 | 51.76 |
| | Gly-4 | | Z.O-2 | | | | | |
| C-27 | 6.67 | 28.93 | 3.33 | 11.01 | 2:1 | 10 | 39.94 | 55.12 |
| C-29 | 3.33 | 14.44 | 6.67 | 22.06 | 1:2 | 10 | 36.50 | 53.34 |
| | Gly-4 | | J.A-3 | | | | | |
| C-30 | 6.67 | 28.93 | 3.33 | 10.32 | 2:1 | 10 | 39.25 | 51.48 |
| C-32 | 3.33 | 14.44 | 6.67 | 20.67 | 1:2 | 10 | 35.12 | 47.35 |
| | Gly-4 | | O.S-2 | | | | | |
| C-33 | 6.67 | 28.93 | 3.33 | 10.55 | 2:1 | 10 | 39.48 | 56.00 |
| C-35 | 3.33 | 14.44 | 6.67 | 21.13 | 1:2 | 10 | 35.57 | 54.99 |

TABLE 27
Percent inhibition in histamine release by the compositions of Glycyrrhiza glabra extract
containing potassium salt of glycyrrhizin (Gly-5) and T. cordifolia ethanol extract (T.C-3)
or Z. officinale oleoresin-1 (Z.O-1) or J. adhatoda ethanol extract (J.A-2)

| | Gly-5 | | T.C-3 | | | | % Inhibition in histamine | |
|---|---|---|---|---|---|---|---|---|
| Comp | | % | | % | | Dose | Additive | |
| # | μg/mL | inhibition | μg/mL | inhibition | Ratio | μg/mL | (Calculated) | Observed |
| C-36 | 6.67 | 28.61 | 3.33 | 11.53 | 2:1 | 10 | 40.13 | 51.29 |
| C-38 | 3.33 | 14.28 | 6.67 | 23.09 | 1:2 | 10 | 37.37 | 49.61 |
| | Gly-5 | | Z.O-1 | | | | | |
| C-39 | 6.67 | 28.61 | 3.33 | 10.64 | 2:1 | 10 | 39.24 | 49.07 |
| C-41 | 3.33 | 14.28 | 6.67 | 21.30 | 1:2 | 10 | 35.58 | 48.03 |
| | Gly-5 | | J.A-2 | | | | | |
| C-42 | 6.67 | 28.61 | 3.33 | 9.71 | 2:1 | 10 | 38.32 | 48.41 |
| C-44 | 3.33 | 14.28 | 6.67 | 19.45 | 1:2 | 10 | 33.73 | 44.61 |

### Example 38: In-Vivo Study of the Airway Allergic Inflammation and Immune Modulation in Ovalbumin-Induced C57BL/6 Mice

Decrease in total Leucocytes, Neutrophils, Lymphocytes, and Eosinophils: Modulations of airway allergic inflammation and immune response by the test samples were evaluated in ovalbumin (OVA, Sigma Chemicals, USA) induced 8-9 weeks old (20-25 g body weight) male C57BL/6 mice. The animals were acclimatized for five days, and then they were randomized and allocated into Group-1 (G1) to Group-5 (G5) based on their body weights (BW). Each group contained eight animals (n=8). Mice in G1 and G2 received Carboxymethylcellulose (CMC) as a vehicle, and G3-G5 received 100 mg per kg BW of T.C-1, Gly-2, and comp-45 mixed with CMC through oral gavage for 12 days. On day 1, each mice in G2-G5 was sensitized with 100 μg OVA in 200 μL alum adjuvant via the intraperitoneal route. These animals were given intranasal challenges with 100 μg OVA (in 50 μL sterile 1×PBS) on day 8, and 50 μg OVA (in 50 L 1×PBS) on days 10 and 12 under mild anesthesia. The G1 or vehicle control animals received equivalent volumes of sterile PBS on the days of OVA sensitization and challenges. On day 12, blood samples were collected under mild anesthesia for estimation of serum interferon-gamma (IFN-γ) and immunoglobulin E (IgE). Post blood collection, respective animals were euthanized using an overdose of thiopentone sodium, followed by exsanguination, and subjected to necropsy. Bronchoalveolar lavage fluid (BALF), spleen, and lung tissues were collected. Total Leucocyte Count (TLC) and Differential Leucocyte Count (DLC) (neutrophils, lymphocytes, and eosinophil) were performed from BALF; the spleen was processed for analysis of CD4 and CD8 cells using flow cytometry. One lobe of the lung tissue was fixed in formalin for gross pathology and microscopic examinations.

Serum biomarker analysis (IFN-γ and IgE): The levels of IFN-γ and IgE in the rat serum samples were analyzed using enzyme-linked immunosorbent assay (ELISA) kits. Briefly, 100 μL of respective samples/standards were added to respective wells of the pre-coated 96-well ELISA plates and incubated for 2.5 hours at room temperature. After the incubation, the plates were washed with 1× wash buffer; 100 μL of 1× prepared detection antibody was added and incubated for 1 hour at room temperature with gentle shaking. After washing the wells, 100 μL Streptavidin solution was added; the plate was sealed and incubated for 45 minutes at room temperature with gentle shaking. After wash, TMB substrate was added; plates were sealed and incubated for 30 minutes in the dark at room temperature with gentle shaking. In each well, fifty microliters of stop solution were added, and absorbance was measured at 450 nm using a microplate reader (Spectramax2e, Molecular Devices, San Jose, CA). The levels of IFN-γ and IgE were quantified utilizing the respective standard curves generated for the respective analyte.

CD4 and CD8 cell population analysis in splenocytes: The spleens of the experimental mice were gently crushed on Falcon® 100 μm sterile cell strainers (Corning, USA) using 1×RPMI medium to obtain single cells suspension of splenocytes. The splenocytes were counted, and 0.3×10⁶ splenocytes suspended in FACS buffer were taken into each well of a ‘v’ bottom 96-well plate and processed for flow cytometry staining. The cells were washed with FACS buffer and incubated 70 ng of PE anti-mouse CD4 Antibody (BioLegend, USA) and 70 ng of APC anti-mouse CD8a Antibody (BioLegend, USA) for 30 minutes in the dark at room temperature. After the incubation, the cells were washed with FACS buffer and fixed using 100 μL of BD Cytofix™ Fixation Buffer (BD Biosciences, USA) for 20 minutes in the dark. After incubation, the cells were washed with FACS buffer and resuspended in the same buffer and acquired on BD FACSVerse flow cytometer for analysis. Percent positive CD4+ and CD8+ populations were recorded.

Histopathology examinations: The lung tissues were fixed in 0% neutral buffered formalin for 48 hours in the dark at room temperature. The fixed tissues were embedded in paraffin and the paraffin-embedded tissues were sectioned at 4-micron sizes in a rotary microtome. The tissue sections were stained with hematoxylin-eosin and toluidine blue to examine the inflammatory cells and mast cells, respectively. The stained tissue sections were examined under 10× objective of a light microscope (Axio scope Al, Carl Zeiss, Germany). For each parameter, one hundred random fields were examined. The results are presented in Tables: 28-32.

TABLE 28
Percent decrease in total Leucocytes and Neutrophils count in
bronchoalveolar lavage (BAL) fluid of the experimental mice
supplemented with G. glabra extract containing zinc/potassium
double salt of glycyrrhizin (Gly-2), T. cordifolia water
extract (T.C-1), or their composition in 2:1 ratio (Comp-45)

| | Total Leucocytes | | Neutrophils | |
|---|---|---|---|---|
| | | % decrease | Differential | % decrease |
| | | from | count % | from |
| Experimental | Count | OVA- | of total | OVA- |
| group | (10³/mL) | induced | Lucocytes) | induced |
| Vehicle control | 0.6 | — | 2.2 | — |
| (G1) | | | | |
| OVA-induced | 1.7 | — | 22.4 | — |
| (G2) | | | | |
| OVA + T.C-1 | 1.2 | 30.3 | 17.1 | 23.5 |
| (G3) | | | | |
| OVA + Gly-2 | 1.1 | 36.4 | 14.6 | 34.6 |
| (G4) | | | | |
| OVA + Comp-45 | 0.8 | 54.5 | 9.8 | 56.4 |
| (G5) | | | | |

TABLE 29
Percent decrease in Lymphocytes and Eosinophil count in
bronchoalveolar lavage (BAL) fluid of the experimental mice
supplemented with G. glabra extract containing zinc/potassium
double salt of glycyrrhizin (Gly-2), T. cordifolia water
extract (T.C-1), or their composition in 2:1 ratio (Comp-45)

| | Lymphocytes | | Eosinophils | |
|---|---|---|---|---|
| | Differential | % | Differential | % |
| | count | decrease | count | decrease |
| | (% of | from | (% of | from |
| Experimental | total | OVA- | total | OVA- |
| group | Leucocytes) | induced | Leucocytes) | induced |
| Vehicle control | 3.8 | — | 0.3 | — |
| (G1) | | | | |
| OVA-induced | 5.6 | — | 5.5 | — |
| (G2) | | | | |
| OVA + T.C-1 | 4.6 | 17.8 | 3.3 | 40.9 |
| (G3) | | | | |
| OVA + Gly-2 | 4.4 | 22.2 | 2.9 | 47.7 |
| (G4) | | | | |
| OVA + Comp-45 | 4.0 | 28.9 | 2.0 | 63.6 |
| (G5) | | | | |

TABLE 30
Percent increase in serum interferon-gamma (IFN-γ) and
percent decrease in serum IgE of the experimental mice
supplemented with G. glabra extract containing zinc/
potassium double salt of glycyrrhizin (Gly-2), T. cordifolia
water extract (T.C-1), or their composition in 2:1 ratio (Comp-45)

| | Serum IFN-γ | | Serum IgE | |
|---|---|---|---|---|
| | Mean | % increase | Mean | % decrease |
| Experimental | concn | from OVA- | concn | from OVA- |
| group | (pg/mL) | induced | (mg/mL) | induced |
| Vehicle control | 37.0 | — | 0.4 | — |
| (G1) | | | | |
| OVA-induced | 25.2 | — | 1.9 | — |
| (G2) | | | | |
| OVA + T.C-1 | 28.8 | 14.0 | 1.7 | 10.8 |
| (G3) | | | | |
| OVA + Gly-2 | 29.7 | 17.6 | 1.6 | 15.0 |
| (G4) | | | | |
| OVA + | 31.3 | 24.2 | 1.2 | 38.5 |
| Comp-45 | | | | |
| (G5) | | | | |

TABLE 31
Percent decrease in inflammatory cells and mast cells count
in the lungs of the experimental mice supplemented
with G. glabra extract containing zinc/potassium double salt
of glycyrrhizin (Gly-2), T. cordifolia water extract (T.C-
1), or their composition in 2:1 ratio (Comp-45)

| | Total | | | |
|---|---|---|---|---|
| | inflammation score | | | |
| | (peribronchial + | | | |
| | perivascular | | | |
| | PMN cells) | | Mast cells | |
| | | % decrease | | % decrease |
| Experimental | Mean | from OVA- | Mean | from OVA- |
| group | count | induced | count | induced |
| Vehicle control | 0.0 | — | 1.2 | — |
| (G1) | | | | |
| OVA-induced (G2) | 3.9 | — | 2.5 | — |
| OVA + T.C-1 (G3) | 2.8 | 29.0 | 2.1 | 15.0 |
| OVA + Gly-2 (G4) | 2.4 | 38.7 | 1.8 | 30.0 |
| OVA + Comp-45 | 1.9 | 51.6 | 1.5 | 40.0 |
| (G5) | | | | |

TABLE 32
Percent increase in CD4+ and CD8+ T-cell subsets in
the spleen of the experimental mice supplemented
with G. glabra extract containing zinc/potassium double salt of
glycyrrhizin (Gly-2), T. cordifolia water extract (T.C-1), or their
composition in 2:1 ratio (Comp-45)

| | T cell subsets in spleen | | | |
|---|---|---|---|---|
| | Mean | | Mean | |
| | population | % increase | population | % increase |
| Experimental | of CD4+ | from OVA- | of CD8+ | from OVA- |
| group | cells (%) | induced | cells (%) | induced |
| Vehicle control | 20.0 | — | 10.4 | — |
| (G1) | | | | |
| OVA-induced | 15.7 | — | 10.0 | — |
| (G2) | | | | |
| OVA + T.C-1 | 16.4 | 4.8 | 10.1 | 0.9 |
| (G3) | | | | |
| OVA + Gly-2 | 17.2 | 10.1 | 10.3 | 3.4 |
| (G4) | | | | |
| OVA + Comp-45 | 18.4 | 17.6 | 10.7 | 7.6 |
| (G5) | | | | |

It is evident from the above experimental data that the compositions (C-1 to C-44) of the present invention unexpectedly showed synergistic efficacy in increasing the production of IFN-γ (tables 8-12) and IL-2 (tables 13-17) and in inhibiting the production of LTB₄(Tables 18-22); and in inhibiting Histamine release (tables 23-27) when compared to their corresponding individual ingredients. Further it also well established that the compositions (C-1 to C-44) of the present invention unexpectedly showed synergistic efficacy in decreasing the total Leucocytes and Neutrophils (table 28); decreasing in Lymphocytes and Eosinophil (table 29) count in bronchoalveolar lavage (BAL) fluid; increasing serum interferon-gamma (IFN-γ) production and decreasing serum IgE (table 30) in experimental mice when compared to individual ingredients. Furthermore, it is demonstrated by the present inventors that the compositions (C-1 to C-44) of the present invention decreases inflammatory cells and mast cells count in the lungs (table 31) and increases the count in CD4+ and CD8+ T-cell subsets (table 32) in the spleen of the experimental mice.

Therefore, the synergistic compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; are useful for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

1. Synergistic herbal compositions comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; for obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose.

2. The synergistic herbal compositions as claimed in claim-1, wherein the concentration of Glycyrrhiza glabra extract containing metal salt varies in the range of 10%-90% by weight and the concentration of the extract, fraction, phytochemical or mixtures thereof of the second ingredient selected from Tinospora cordifolia or Zingiber officinale or Justicia adhatoda or Ocimum sanctum; varies in the range of 90%-10% by weight.

3. The synergistic herbal compositions as claimed in claim-1, wherein the metal salt(s) of glycyrrhizin consists glycyrrhizin in the range of 2.0-50% and zinc in the range of 0.1-5.0% and potassium in the range of 0.1-10.0%.

4. The synergistic herbal compositions as claimed in claim-1, wherein the extract, fraction or mixtures thereof derived from Tinospora cordifolia, Zingiber officinale, Justicia adhatoda, and Ocimum sanctum; are standardized to at least one phytochemical reference marker compound or pharmacologically active marker; wherein phytochemical marker compound or group of phytochemical compounds is in the concentration range of 0.01% to 90% by weight of the extract.

5. The synergistic herbal compositions as claimed in claim-1, wherein the extracts or fractions or phytochemicals or mixtures thereof are obtained from at least one plant part selected from the group comprising leaves, stems, tender stems, tender twigs, aerial parts, whole fruit, fruit peel rind, seeds, flower heads, root, bark, hardwood, rhizome or whole plant or mixtures thereof.

6. The synergistic herbal compositions as claimed in claim-1, wherein the extract, fraction, phytochemical or mixtures thereof; are produced using at least one solvent selected from C1-C5 alcohols selected from ethanol, methanol, n-propanol, isopropyl alcohol; ketones selected from acetone, methylisobutyl ketone, chlorinated solvents selected from methylene dichloride and chloroform; water and mixtures thereof; C1-C7 hydrocarbons such as hexane; esters like ethyl acetate and the like and mixtures thereof.

7. The synergistic herbal compositions as claimed in claim-1, further contain optionally at least one component selected from pharmaceutically or nutraceutically or dietically acceptable excipients, carriers, and diluents.

8. The synergistic herbal compositions as claimed in claim-7, wherein the pharmaceutically or nutraceutically or dietically acceptable excipients, carriers and diluents are selected from monosaccharide's such as glucose, dextrose, fructose, galactose etc.; disaccharides such as but not limited to sucrose, maltose, lactose, lactulose, trehalose cellobiose, chitobiose, Glucidex 12D etc.; polycarbohydrates such as starch and modified starch such as sodium starch glycolate, pre-gelatinized starch, soluble starch, and other modified starches, ultrasperse A; dextrins that are produced by hydrolysis of starch or glycogen such as yellow dextrin, white dextrin, maltodextrin etc.; polyhydric alcohols or sugar alcohols such as but not limited to sorbitol, mannitol, inositol, xylitol, isomalt etc.; cellulose based derivatives such as but not limited to microcrystalline cellulose, hydroxy propyl methyl cellulose, hydroxy ethyl cellulose etc.; silicates such as but not limited to neusilin, veegum, talc, colloidal silicon dioxide, syloid etc.; metallic stearates such as but not limited to calcium stearate, magnesium stearate, zinc stearate etc.; organic acids such as citric acid, tartaric acid, malic acid, succinic acid, lactic acid, L-ascorbic acid etc.; fatty acid esters and esters of poly sorbate, natural gums such as but not limited to acacia, carrageenan, guar gum, xanthan gum etc.; vitamin B group, nicotinamide, calcium pantothenate, amino acids, proteins such as but not limited to casein, gelatin, pectin, agar; organic metal salts such as but not limited to sodium chloride, calcium chloride, dicalcium phosphate, zinc sulphate, zinc chloride etc.; natural pigments, flavors, class I & class II preservatives and aqueous, alcoholic, hydro-alcoholic, organic solutions of above listed ingredients alone or in combination.

9. The synergistic herbal compositions as claimed in claim 1, wherein the composition is formulated into a dosage form selected from dry powder form, liquid form, beverage, food product, dietary supplement, or any suitable form such as a tablet, a capsule, a soft chewable tablet or gummy bear.

10. The synergistic herbal compositions as claimed in claim 1, wherein the composition is formulated into nutritional/dietary supplements that can be contemplated/made into the dosage form of healthy foods, or food for specified health uses such as solid food like chocolate or nutritional bars, semisolid food like cream, jam, or gel or beverage such as refreshing beverage, lactic acid bacteria beverage, drop, candy, chewing gum, gummy candy, yogurt, ice cream, pudding, soft adzuki bean jelly, jelly, cookie, tea, soft drink, juice, milk, coffee, cereal, snack bar.

11. The synergistic herbal compositions as claimed in claim 1, wherein the composition is formulated into controlled-release tablets, using controlled release polymer-based coatings by the techniques including nanotechnology, microencapsulation, colloidal carrier systems and other drug delivery systems for obtaining the desired therapeutic benefit.

12. A process for preparing synergistic herbal compositions comprising a first ingredient Glycyrrhiza glabra extract containing at least one metal salt of glycyrrhizin selected from zinc salt of glycyrrhizin, the potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally contains at least one component selected from pharmaceutically or nutraceutically or dietically acceptable excipients, carriers and diluents; wherein the process comprises the following steps of;
(i) extracting dried Glycyrrhiza glabra root powder with suitable solvent;
(ii) optionally passing the extract solution from step (i) through a cation exchange resin;
(iii) neutralizing the solution of step (i) or step (ii) with metal compound(s);
(iv) filtering the solution;
(v) evaporating the solvent and drying the residue to obtain Glycyrrhiza glabra extract containing metal salt of glycyrrhizin;
(vi) blending Glycyrrhiza glabra extract containing metal salt of glycyrrhizin in step (v) with at least one extract derived from Tinospora cordifolia, Zingiber officinale, Justicia adhatoda, and Ocimum sanctum;
optionally in the presence of pharmaceutically or nutraceutically or dietically acceptable excipients, carriers, and diluents; and
(vii) drying the product under a vacuum to get the compositions.

13. The process for preparing synergistic compositions as claimed in claim-12, wherein the suitable solvent used in the process for the preparation of the extracts or fractions or phytochemicals or mixtures thereof are produced by using at least one solvent selected from; C1-C5 alcohols selected from ethanol, methanol, n-propanol, isopropyl alcohol; ketones selected from acetone, methylisobutyl ketone; chlorinated solvents selected from methylene dichloride and chloroform; water and mixtures thereof; C1-C7 hydrocarbons such as hexane; esters like ethyl acetate and the like and mixtures thereof.

14. The process for preparing synergistic compositions as claimed in claim-12, wherein the metal is selected from zinc and potassium; and the metal compound used for the preparation of these compositions is in the form of their metal salts, metal oxides, metal hydroxides or carbonates; selected but not limited to zinc oxide, zinc carbonate, zinc hydroxide, potassium hydroxide, and potassium carbonate.

15. The process for preparing synergistic compositions as claimed in claim-12, wherein the cation exchange resin is selected from but not limited to Tulson T 42, Tulson CXO 12, Dowex marathon, Indion 225, Indion 236, Indion 730, Indion 652, Diaion SK1B, Diaion PK216, C 100, C 104, C 107, C 800 LT, C 800 MP, C 145, CG 10, D 001, Amberlite IRC 86, WK 11, WK 40L and D113.

16. Method of obtaining at least one health benefit selected from improving immunity/eliciting immune response/rejuvenating the immune system; improving symptoms associated with innate immunity, adaptive immunity, cellular immunity and humoral immunity; strengthening natural defense, preventing viral respiratory infections, improving lung function, supporting respiratory health, and treating/alleviating symptoms associated with airway inflammation and allergic rhinitis that include cold, cough, runny, and itchy nose in a human; wherein the method comprises supplementing human with an effective dose of a herbal composition comprising a first ingredient Glycyrrhiza glabra extract containing at least one metal salt of glycyrrhizin selected from zinc salt of glycyrrhizin, potassium salt of glycyrrhizin, zinc/potassium double salt of glycyrrhizin and mixtures thereof; and a second ingredient selected from extract, fraction, phytochemical or mixtures thereof derived from Tinospora cordifolia, Zingiber officinale, Justicia adhatoda, and Ocimum sanctum; optionally containing at least one component selected from pharmaceutically or nutraceutically or dietically acceptable excipients, carriers and diluents.

17. (canceled)
